(19) United States
(12) Patent Application Publication
(10) Pub. No.: US 2005/0118263 A1
(43) Pub. Date: Jun. 2, 2005

(54) Composition

(76) Inventors: Brian Robert Walker, Edinburgh (GB); Jonathan Robert Seckl, Edinburgh (GB)

Correspondence Address: FROMMER LAWRENCE & HAUG, 745 FIFTH AVENUE- 10TH FL., NEW YORK, NY, 10151, US

(21) Appl. No.: 10/958,549

(22) Filed: Oct. 5, 2004

Related U.S. Application Data
(63) Continuation-in-part of application No. PCT/GB03/01400, filed on Mar. 31, 2003.
(60) Provisional application No. 60/375,690, filed on Apr. 26, 2002.

(30) Foreign Application Priority Data
Apr. 5, 2002 (GB) 0207945.7

(51) Int. Cl. A61K 31/59; A61K 31/4965; A61K 9/22

(52) U.S. Cl. 424/468; 514/171; 514/255.06

(57) ABSTRACT

We provide a composition comprising a first agent which is an antagonist of 11β-HSD1, together with a second agent comprising a diuretic. The second agent may comprise a molecule which is capable of modulating an interaction between the first agent and 11βHSD2. Such a composition may be used for improving cognitive ability of an individual, specifically verbal fluency or verbal memory or logical memory (or any combination thereof), or for treatment of Mild Cognitive Impairment (MCI).

### REFERENCE TO RELATED APPLICATIONS

This application is a continuation-in-part of International Patent Application PCT/GB03/01400 filed Mar. 31, 2003 and published as WO 03/086410 on Oct. 23, 2003, which claims priority from Great Britain Patent Application Number 0207945.7 filed Apr. 5, 2002, and U.S. Provisional Application Ser. No. 60/375,690 filed Apr. 26, 2002.

Each of these applications, and each application and patent mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

It is noted that in this disclosure, terms such as “comprises”, “comprised”, “comprising”, “contains”, “containing” and the like can have the meaning attributed to them in U.S. patent law; e.g., they can mean “includes”, “included”, “including” and the like. Terms such as “consisting essentially of” and “consists essentially of” have the meaning attributed to them in U.S. patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. patent law; namely, that these terms are closed ended.

### FIELD

This invention relates to methods of treatment and diagnosis of disease, and molecules and compositions for use in such methods.

### BACKGROUND

Mild Cognitive Impairment (MCI) is an impairment in cognition, specifically memory performance, that is frequently associated with ageing. The degree and type of impairment distinguishes Mild Cognitive Impairment from dementia in that Mild Cognitive Impairment patients exhibit deficits in secondary tests of memory, but perform normally on standard tests measuring other cognitive domains. Thus, Mild Cognitive Impairment is defined as a clinical disorder that is distinct from early stages of dementia, particularly Alzheimer's type dementia, and can therefore be specifically targeted for treatment intervention.

The underlying causes of memory loss in Mild Cognitive Impairment have not been determined, thus a strategy for treatment has not been easily identified. Although some investigators believe that most Mild Cognitive Impairment patients have neuropathology that is characteristic of Alzheimer's disease, many patients diagnosed with Mild Cognitive Impairment typically do not progress to Alzheimer's Disease, thereby suggesting that Mild Cognitive Impairment has an underlying pathophysiology that is divergent from that of Alzheimer's despite other characteristics that may be shared.

A number of treatments for Alzheimer's disease have been proposed, but there is no consensus regarding the etiology of the disease and it is not clear which, if any, of these treatments would also be effective for Mild Cognitive Impairment. Proposed treatments include the use of various agents such as cholinergic agonists (Asthana et al., Clin. Pharmacol. Ther. 60: 76-282,1996), estrogen, Vitamin E (a-tocopherol), nerve growth factors, or calcium blockers to improve memory or slow the rate of neuronal degeneration and death.

Alternatively, Alzheimer's disease has been hypothesized to be an inflammatory disease similar to an autoimmune disease and the administration of anti-inflammatory agents has been proposed as a therapy. Ongoing clinical studies based on this hypothesis include those using prednisone, a synthetic cortisol agonist (see, e.g., Aisen, Drugs Aging 12: 1-6, 1998; Aisen, Gerontology 43: 143-149,1997; and Aisen, Mol. Chem. Neuropathol. 28: 8388,1996). In apparent contrast to the latter theory, it has also been observed that patients with dementia can exhibit markedly increased levels of the physiological glucocorticoid cortisol (hydrocortisone) (see, e.g., Davis et al, Am. J. Psych. 143: 3,1986; Maeda et al., Neurobiol Aging 12: 161-163,1991). Moreover, it has been suggested that increased glucocorticoid levels may play a role in pathogenesis.

International Patent Application PCT/US00/32260 (published as WO0137840) describes use of agents which inhibit the binding of cortisol to its receptors in methods for treating mild cognitive impairment. Furthermore, International Patent Application PCT/GB96/02134 (published as WO97/07789) describes the use of inhibitors of 11-βHSD1 for treatment of various disorders, including neuronal dysfunction or loss and cognitive impairment. This document incorporates by reference the disclosure of each of these applications.

### SUMMARY

Surprisingly, it has now been found that other molecules, in particular, diuretics, particularly antikaliuretic-diuretics, may be used in conjunction with antagonists or inhibitors of 11β-HSD1, to achieve an improvement in cognitive abilities of individuals. In particular, the combinations disclosed here may be used to improve cognition in individuals suffering from Mild Cognitive Impairment.

According to a first aspect of the present invention, we provide a composition comprising a first agent comprising an antagonist of 11β-HSD1, together with a second agent comprising an anti-kaliuretic-diuretic.

Preferably, the first agent comprises an inhibitor of 11β-HSD1 transcription, translation, expression, synthesis or activity, or in which the first agent is capable of lowering levels of 11β-HSD1. Preferably, the first agent is selected from the group consisting of: carbenoxolone, 11-oxoprogesterone, 3α,17,21-trihydoxy-5β-pregnan-3-one, 21-hydroxy-pregn-4-ene-3,11,20-trione, androst-4-ene-3,11,20-trione and 3β-hydroxyandrost-5-en-17-one.

Preferably, the first agent comprises carbenoxolone. Preferably, the second agent is capable of modulating an interaction between the first agent and 11β-HSD2, preferably capable of down-regulating an antagonistic effect of the first agent on 11β-HSD2.

Preferably, the second agent is not capable of binding to a mineralocorticoid receptor. Preferably, the second agent is capable of preventing renal mineralocorticoid excess. Preferably, the second agent comprises a pyrazine-carbonyl-guanidine.

Preferably, the second agent comprises amiloride (3,5-diamino-6-chloro-N-(diaminomethylene)(pyrazinecarboxamide), or a salt or ester thereof, preferably amiloride-HCl, more preferably amiloride-monohydrochloride, dihydrate. Preferably, the second agent comprises an aldosterone antagonist. Preferably, the second agent comprises an androstadiene-spiro-furan. Preferably, the second agent comprises spironolactone (17-hydroxy-7alpha-mercapto-3-oxo-17alpha-pregn-4-ene-21-carboxylic acid gamma-lactone) or a salt or ester thereof, preferably spironolactone-acetate, or Eplerenone.

There is provided, according to a second aspect of the present invention, a pharmaceutical composition comprising a composition as described, together with a pharmaceutically acceptable carrier, excipient or diluent.

We provide, according to a third aspect of the present invention, a composition as described, which is provided in a slow-release formulation.

As a fourth aspect of the present invention, there is provided a composition as described for use in a method of improving verbal fluency, verbal memory or logical memory, or any combination thereof, in an individual.

As a fifth aspect of the present invention, there is provided a composition as described for use in a method of treatment or prevention of mild cognitive impairment (MCI) in an individual.

In highly preferred embodiments, the individual is suffering from Type 2 diabetes.

The present invention, in a sixth aspect, provides a first agent comprising an antagonist of 11β-HSD1 for use in a method of improving verbal fluency, verbal memory or logical memory, or any combination thereof, in an individual, in which the method comprises administering an 11β-HSD1 antagonist simultaneously or sequentially with a second agent comprising an anti-kaliuretic-diuretic.

In a seventh aspect of the present invention, there is provided a second agent comprising an anti-kaliuretic-diuretic for use in a method of improving verbal fluency, verbal memory or logical memory, or any combination thereof, in an individual, in which the method comprises administering an anti-kaliuretic-diuretic simultaneously or sequentially with a first agent comprising an antagonist of 11β-HSD1.

According to an eighth aspect of the present invention, we provide a first agent comprising an antagonist of 11β-HSD1 for use in a method of treatment or prevention of Mild Cognitive Impairment (MCI) in an individual, in which the method comprises administering an 11β-HSD1 antagonist simultaneously or sequentially with a second agent comprising an anti-kaliuretic-diuretic.

We provide, according to a ninth aspect of the invention, a second agent comprising an anti-kaliuretic-diuretic for use in a method of treatment or prevention of Mild Cognitive Impairment (MCI) in an individual, in which the method comprises administering an anti-kaliuretic-diuretic simultaneously or sequentially with a first agent comprising an antagonist of 11β-HSD1.

There is provided, in accordance with a tenth aspect of the present invention, use of a first agent comprising an antagonist of 11β-HSD1, together with a second agent comprising an anti-kaliuretic-diuretic, for the preparation of a composition for improvement of verbal fluency, verbal memory, or logical memory, or any combination thereof.

As an eleventh aspect of the invention, we provide use of a first agent comprising an antagonist of 11β-HSD1, together with a second agent comprising an anti-kaliuretic-diuretic, for the preparation of a composition for the treatment or prevention of Mild Cognitive Impairment (MCI).

Preferably, the first agent has the features as described. Preferably, the second has the features as described.

Preferably, verbal fluency is significantly improved as assessed by a Controlled Word Association test, or in which verbal memory is significantly improved as assessed by a Rey Auditory-Verbal Learning Test, or in which logical memory is significantly improved as assessed by a Wechsler Memory Scale.

We provide, according to a twelfth aspect of the invention, a kit comprising a first agent comprising an antagonist of 11β-HSD1, and a second agent comprising an anti-kaliuretic-diuretic.

Preferably, the first agent and the second agent are in separate containers. Preferably, the first agent has the features as described. Preferably, the second has the features as described.

The kit may further comprise instructions for administration of the agents to an individual to improve verbal fluency, verbal memory, or logical memory, or any combination thereof. The kit may alternatively, or in addition, comprise instructions for administration of the agents to an individual with mild cognitive impairment.

According to a thirteenth aspect of the present invention, we provide a method of preparing a composition as described, the method comprising admixing a first agent comprising an antagonist of 11β-HSD1, with a second agent comprising an anti-kaliuretic-diuretic. Preferably, the first agent has the features as described. Preferably, the second has the features as described.

There is provided, according to a fourteenth aspect of the present invention, use of a composition as described, for improving verbal fluency, verbal memory or logical memory, or any combination thereof, in an individual.

We provide, according to a fifteenth aspect of the present invention, use of a composition as described, for treating or preventing Mild Cognitive Impairment in an individual.

According to a sixteenth aspect of the present invention, we provide a method of improving any one or more of verbal fluency, verbal memory or logical memory, or any combination thereof, in an individual, which method comprises administering to an individual a first agent comprising an antagonist of 11β-HSD1, simultaneously or sequentially with a second agent comprising an anti-kaliuretic-diuretic.

According to a seventeenth aspect of the present invention, we provide a method of treatment or prevention of mild cognitive impairment (MCI) in an individual, which method comprises administering to an individual a first agent comprising an antagonist of 11β-HSD1, simultaneously or sequentially with a second agent comprising an anti-kaliuretic-diuretic.

Preferably, the first agent has the features as described. Preferably, the second has the features as described. Preferably, the method comprises administering to an individual a therapeutically effective amount of a composition as described. The first agent may be administered at a rate of about 4.5 mg/kg/day. The second agent may be administered at a rate of about 0.15 mg/kg/day. In preferred embodiments, the individual is suffering from Type 2 diabetes.

A pharmaceutically or therapeutically effective amount is an amount of a composition which achieves the desired effect in an animal, human or individual. The actual amount will vary on a number of factors, as known to those skilled in the art. Using the guidance given herein and knowledge of the art, the determination of a pharmaceutically effective amount is within the ordinary skill of a physician. Pharmaceutically effective amounts designed for particular applications may be packaged as unit doses to facilitate administration.

We provide, according to an eighteenth aspect of the present invention, use of a composition as described, for any one or more of the purposes as set out in Table 2.

According to other aspects of the invention, we provide a method of treatment of a human or animal patient suffering from a condition selected from the group consisting of: hepatic insulin resistance, adipose tissue insulin resistance, muscle insulin resistance, neuronal loss or dysfunction due to glucocorticoid potentiated neurotoxicity, obesity and any combination of the aforementioned conditions, the method comprising the step of administering to said patient a medicament comprising a pharmaceutically active amount of a first agent which is an antagonist of 11β-HSD1, simultaneously or sequentially with a second agent which comprises a diuretic, preferably an antikaliuretic-diuretic.

### BRIEF DESCRIPTION OF THE FIGURES

FIG. 1 shows expression of 11βHSD1 mRNA in human brain. The image of “Hippocampal neurons” is a microscopic view showing 11β-HSD1 mRNA as silver grains over an identified hippocampal neuron (arrow; CA3 subfield) counterstained with haematoxylin (nuclei). Note the black silver grains cluster over the cell bodies

FIG. 2 is a graph showing percentage increase in cognitive performance on administration of a mixture of carbenoxolone and amiloride.

### DETAILED DESCRIPTION

This invention is based on the surprising discovery that use of an agent capable of antagonising 11β-HSD1, in combination with a diuretic, is effective for improving cognitive abilities in individuals. In particular, we find that such a combination is useful in treating Mild Cognitive Impairment. In addition, we have found that such a combined treatment is safe. We have found that giving carbenoxolone alone resulted in 3 withdrawals in 8 patients with type 2 diabetes, due to sodium retention (including one hospital admission with hypokalaemia) while giving it with amiloride has resulted in no withdrawals).

We also find that administration of an agent capable of antagonising 11β-HSD1, in combination with a diuretic, to patients suffering from Type 2 diabetes increases their cognitive abilities (see Example 3). Accordingly, the methods and compositions described here are useful for improving cognitive abilities in Type 2 diabetes individuals.

Particularly effective treatments comprise use of an antikaliuretic-diuretic. In improving cognitive ability and treating Mild Cognitive Impairment, the methods and compositions described here can preferably improve the impairment of memory, and/or, the rate of, or extent of, any further decline in memory function. Furthermore, preferably, the methods and compositions described here are effective in improving verbal memory, verbal fluency, logical memory or memory performance, or preventing or slowing further memory impairment, preferably in an Mild Cognitive Impairment patient (whether or not suffering from Type 2 diabetes).

Thus, while the methods and compositions described here are particularly suitable for improvements in individuals suffering from Mild Cognitive Impairment, they may also suitably be used for improving cognitive abilities of “normal” individuals (i.e., those which are not suffering from Mild Cognitive Impairment). Such individuals may include those suffering from Type 2 diabetes. Thus, the methods and compositions described here are found to lead to improvements in verbal fluency and/or verbal memory, and/r logical memory, for patients suffering from Mild Cognitive Impairment, as well as for normal individuals.

Accordingly, we provide the use of a first and second agent, preferably in the form of a composition comprising both, for these purposes. In preferred embodiments, the first agent comprises an antagonist of 11β-HSD1, and the second agent which comprises a diuretic, preferably an anti-kaliuretic-diuretic. Suitably, the second agent is one which is capable of modulating an interaction between the first agent and 11β-HSD2.

The first and second agent preferably independently comprise one, some or all of the following activities: diuretic activity, sodium diruretic activity, anti-kaliuretic diuretic activity, anti-aldosterone activity, anti-hypertensive activity, anti-androgenic activity and positive inotrope activity. In preferred embodiments, the first and second agent independently comprise one, some or all of the following activities: potassium-sparing diuretic activity, anti-kaliuretic diuretic activity an anti-aldosterone activity. In highly preferred embodiments, only the second agent comprises any or all of the listed activities.

The term “treating” refers to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being; or, in some situations, preventing the onset of dementia.

The treatment or amelioration of symptoms can be based on objective or subjective parameters; including the results of a physical examination, neuropsychiatric exams, and/or a psychiatric evaluation. For example, the methods and compositions described here successfully treat a patient's Mild Cognitive Impairment by improving performance of memory task tests and/or slowing or preventing the rate of, or extent of, cognitive decline.

“Expression”, as in gene expression, is used herein to refer to the process of transcription and translation of a gene to produce a gene product, be it RNA or protein. Thus, inhibition of expression may occur at any one or more of many levels, including transcription, post-transcriptional processing, translation, post-translational modification, and the like. Agents which modulate gene expression, including transcription or translation, include for example agents which downregulate or knock out endogenous genes; including agents which knock out genes in pluripotent cells which give rise to all or part of an animal.

Inhibition of 11β-HSD1 “synthesis or activity” refers to the inhibition of 11β-HSD1 at the protein level, to prevent or downregulate the production of the protein, or at least one biological activity of the protein once produced.

The methods and compositions described here may also be used for other purposes, as described below. For example, they may be used to reduce the risk of an individual contracting cardiovascular disease. “Cardiovascular disease risk” is the risk, as measured according to accepted risk factors, to which an animal is exposed of suffering from one or more cardiovascular complaints or pathologies. Cardiovascular disease (CVD) includes coronary heart disease (CHD) and stroke. The measurement of risk itself is largely statistical; in the context of the present document, the presence or absence of factors which are accepted to contribute to increasing or decreasing the risk of CVD according to statistical analyses are taken as indicative of increased or decreased risk respectively.

Furthermore, the methods and compositions described here may be effective in establishing an atheroprotective lipid profile in an individual, when administered to him. An “atheroprotective” profile is a profile which prevents, offsets or ameliorates the pathogenesis of atherosclerosis.

The first agent which is an antagonist of 11β-HSD1, and the second agent which comprises an anti-kaliuretic-diuretic, may be administered simultaneously, that is to say, at the same time. For this purpose, a mixture of both agents may be administered, or a separate first agent may be administered together with a separate second agent to the individual at the same time. A composition comprising both agents may be administered to achieve simultaneous administration, or separate compositions, one containing the first agent, and the other containing the second agent, may be administered to the individual at the same time.

The first agent and the second agent may be administered sequentially, that is to say, not at the same time. One agent may be administered, followed by the other. Subsequent administrations of the or each agent may follow. The agents may be alternated, or there may be two or more consecutive administrations of the same agent, at the same or different dosages. Therefore, we envisage regimes such as A1-A2, A2-A1, A1-A2-A1, A2-A1-A2, A1-A2-A1-A2, A2-A1-A2-A1, etc, where A1 is the first agent, and A2 the second agent.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

Mild Cognitive Impairment (MCI)

The term “mild cognitive impairment (MCI)” refers to a category of memory and cognitive impairment that is typically characterised by a clinical dementia rating (CDR) of 0.5 (see, e.g., Hughes et al., Brit. J. Psychiat. 140: 566-572,1982) and further characterised by memory impairment, but not impaired function in other cognitive domains. Memory impairment is preferably measured using tests such as a “paragraph test”. A patient diagnosed with Mild Cognitive Impairment often exhibits impaired delayed recall performance.

Mild Cognitive Impairment is typically associated with ageing and generally occurs in patients who are 45 years of age or older.

The term “dementia” refers to a psychiatric condition in its broadest sense, as defined in American Psychiatric Association: Diagnostic and Statistical Manual of Mental Disorders, Fourth Edition, Washington, D.C., 1994 (“DSM-IV”). The DSM-IV defines “dementia” as characterised by multiple cognitive deficits that include impairments in memory and lists various dementias according to presumed etiology. The DSM-IV sets forth a generally accepted standard for such diagnosing, categorising and treating of dementia and associated psychiatric disorders.

Mild Cognitive Impairment can be manifested as mental or psychological deficits that include impairment in memory, but normal function in other cognitive domains. Thus, a variety of means of diagnosing Mild Cognitive Impairment and assessing the success of treatment, i.e., the success and extent the Mild Cognitive Impairment is treated by the methods and compositions described here, can be used, and a few exemplary means are set forth herein. These means can include classical, subjective psychological evaluations and neuropsychiatric examinations as described below.

The methods and compositions described here include use of a first agent which is an antagonist of 11β-HSD1, and we describe examples of such agents suitable for use. Furthermore, we describe examples of suitable second agents which comprise diuretics and anti-kaliuretic-diuretics in combination with the first agent. However, it will be evident that routine procedures can be used to identify further compounds and compositions capable of exhibiting these properties for use in practising the methods as described here.

Diagnosis of Mild Cognitive Impairment

Mild Cognitive Impairment is characterised as a mild impairment of cognition categorised as a CDR of 0.5 that is associated with deficits in a memory task test, such as a paragraph test. An Mild Cognitive Impairment patient is fully oriented, but demonstrates mild consistent forgetfulness.

Impairment in cognitive domains other than memory, such as problem solving and judgement is doubtful, if present at all, and, further, the Mild Cognitive Impairment patient does not demonstrate impairment in functioning in the community or at home. A patient with Mild Cognitive Impairment scores normally on standard tests of dementia.

There are various means to diagnose the onset of Mild Cognitive Impairment and to assess the efficacy of treatment using the methods and compositions described here. These include the administration of psychiatric tests to determine the CDR, the administration of memory tests, and the administration of psychiatric tests for dementia, which are used to exclude a diagnosis of dementia. The results of these test may be considered in conjunction with other objective physical tests as described below. These means are also useful for assessing the efficacy of the methods and compositions described here in improving memory or decreasing or diminishing further impairment in memory or cognitive decline in a patient with Mild Cognitive Impairment. While the practitioner can use any set of prescribed or empirical criteria that are defined in the scientific and patent literature to diagnose the presence of Mild Cognitive Impairment as an indication to practice the methods and compositions described here, some illustrative diagnostic guidelines and examples of relevant symptoms and conditions are described below. Subjective and objective criteria can be used to measure and assess the success of a particular GR antagonist, pharmaceutical formulation, dosage, treatment schedule or regimen. The features (symptoms) of and criteria for diagnosing Mild Cognitive Impairment are described, e.g., in Petersen et al., Arch. Neurol. 56: 303 308,1999. a. Assessing and diagnosing MCI.

Mild Cognitive Impairment can be diagnosed by formal psychiatric assessment using subjective diagnosis or objective test criteria to determine whether an individual is afflicted with Mild Cognitive Impairment. The methods and compositions described here are preferably practised early in the course of (in the early stages of) Mild Cognitive Impairment, and most preferably, at the first sign of the disease. This is especially critical in the case of Mild Cognitive Impairment patients who may be at risk for progression to Alzheimer's Disease, for example, patients who bear the apolipoprotein E s4 genotype (see, e.g., Tierney et al., Neurology 45: 149-154,1996).

Mild Cognitive Impairment can be diagnosed and evaluated using any of the many objective tests or criteria well-known and accepted in the fields of psychology or psychiatry. Objective tests can used to determine whether an individual is suffering from impaired memory function or dementia and to measure and assess the success of a particular GR antagonist, pharmaceutical formulation, dosage, treatment schedule or regimen. For example, measuring changes in cognitive ability and memory aids in the diagnosis and treatment assessment of a patient Mild Cognitive Impairment. Any test known in the art can be used.

One criterion for the diagnosis of Mild Cognitive Impairment is that the patient receives a CDR of 0.5 as described, e.g., in Hughes et al., Brit. J. Psychiat. 140: 566-572,1982 and Morris, Neurology 43: 2412-2414,1993. In determining the CDR, a patient is typically assessed and rated in each of six cognitive and behavioural categories: memory, orientation, judgement and problem solving, community affairs, home and hobbies, and personal care.

The assessment may include historical information provided by the patient, or preferably, a corroborator who knows the patient well. The patient is assessed and rated in each of these areas and the overall rating, (0,0.5,1.0,2.0 or 3.0) determined. A rating of 0 is considered normal. A rating of 1.0 is considered to correspond to mild dementia. A patient with a CDR of 0.5 is characterised by mild consistent forgetfulness, partial recollection of events and “benign” forgetfulness. The patient is fully oriented and exhibits little impairment in determining similarities and differences and other problem solving skills, or impairment in function in terms of the community, home, or personal care.

Memory Task Test

A hallmark of Mild Cognitive Impairment is impaired performance on a memory task test. Preferably, therefore, the methods and compositions described here provide improved performance on a memory task test, as set out below.

Memory may be measured by such tests known in the art as the Wechsler Memory Scale or a pair-associated memory task. A patient is considered to exhibit impaired performance on such a test if the score is below the education and age-adjusted cut-off for that test. Mild Cognitive Impairment is typically characterised by impairment in delayed recall memory functions, which can be specifically addressed as a component of a memory task test. For example, impaired memory function may be documented by scoring at or below the education cut-off on the Logical Memory II subscale (Delayed Paragraph Recall) from the Wechsler Memory Scale-Revised, of which the maximum score is 25. Age and education-adjusted cut-offs are determined using methods known in the art (see, e.g., Ivnik et al. Clinc. Neuropsychol 6 (Suppl): 1-30 and 49-82,1992; Ivnik et al. J. Consult Clin. Psychol 3: 1991; Ivnik et al., Clin. Neuropsychol. 10: 262-276,1996) An example of these cutoffs are: a) less than or equal to 8 for 16 or more years of eduction; b) less than or equal to 4 for 8-15 years of education and c) less than or equal to 2 for 0-7 year of education. A cutoff value may be determined, for example, by selecting a value that is 1, preferably 1.5, or more standard deviations from the norm for that education and age cohort.

For the purposes of this document, there is an “improvement” in memory, fluency, etc if there is a statistically significant difference in the direction of normality between the performance of patients treated using the methods and compositions described here as compared to members of a placebo group or between subsequent tests given to the same patient. For the purposes of this document, statistical significance is present at a p value of≦0.1, preferably a p value of≦0.05, more preferably a p value of≦0.03, most preferably a p value of≦0.01.

In order to diagnose Mild Cognitive Impairment, a patient must also be categorised as not being demented. Accordingly, a diagnosis of Mild Cognitive Impairment includes neuropychological evaluation for dementia. The criteria for dementia are described, e.g., in the DSM-IV, supra. While the practitioner can use any criteria or means to evaluate dementia, the DSM-IV sets forth a generally accepted standard for such diagnosing, categorising and treating dementia and associated psychiatric disorders, including Alzheimer's disease and multi-infarct dementia.

Several illustrative examples of such criteria utilised in the methods and compositions described here are set forth below.

One objective test for dementia is the so-called Mini-Mental State Examination (MMSE), as described by Folstein “‘Mini-mental state.’A practical method for grading the cognitive state of patients for the clinician.” J. Psychiatr. Res. 12: 189-198, 1975. The MMSE evaluates the presence of global intellectual deterioration. See also Folstein “Differential diagnosis of dementia. The clinical process.” Psychiatr Clin North Am. 20: 45-57,1997. The MMSE is a long-recognised means to evaluate the onset of dementia and the presence of global intellectual deterioration, as seen in Alzheimer's disease and multi-infart dementia. See, e.g., Kaufer, J. Neuropsychiatry Clin. Neurosci. 10: 55-63,1998; Becke, Alzheimer Dis Assoc Disord. 12: 54-57,1998; Ellis, Arch. Neurol. 55: 360-365,1998; Magni, Int. Psychogeriatr. 8: 127-134,1996; Monsch, Acta Neurol. Scand. 92: 145-150,1995. The MMSE is scored from 1 to 30.

The MMSE does not evaluate basic cognitive potential, as, for example, the so-called IQ test. Instead, it tests intellectual skills. A person of “normal” intellectual capabilities will score a “30” on the MMSE objective test (however, a person with a MMSE score of 30 could also score well below “normal” on an IQ test). Accordingly, the methods and compositions described here are appropriately administered when an individual scores 30 on the MMSE. Because it is possible for a “normal” individual to score less than 30 upon a single administration of a test, a “normal” indication on the test is considered to be a score of 30 on at least one test in three administrations of the test.

Another means to evaluate dementia, particularly Alzheimer's disease, is the Alzheimer's Disease Assessment Scale (ADAS-Cog), or a variation termed the Standardized Alzheimer's Disease Assessment Scale (SADAS). It is commonly used as an efficacy measure in clinical drug trials of Alzheimer's disease and related disorders characterised by cognitive decline. SADAS and ADAS-Cog were not designed to diagnose Alzheimer's disease; they are useful in characterising symptoms of dementia and are a relatively sensitive indicator of dementia progression. (See, e.g., Doraiswamy, Neurology 48: 1511-1517,1997; and Standish, J. Am. Geriatr. Soc. 44: 712-716,1996.) The evaluation for the presence of Mild Cognitive Impairment can also utilise a combination of subjective diagnosis and objective testing. For example, family history and history provided by the patient as well as other individuals can be used as a component in the determination of Mild Cognitive Impairment.

Other tests may also be considered in diagnosing Mild Cognitive Impairment. In one study (Petersen et al., Arch Nuerol. 56: 303-308,1999), patients were seen by a behavioural neurologist who obtained a medical history from the patients and corroborating sources, and performed a variety of tests including the Short Test of Mental Status, Hachinski Ischemic Scale, and a neurologic examination. Other data collected included the Record of Independent Living, Geriatric Depression Scale, and additional family history information as well as laboratory tests such as a chemistry group, complete blood cell count, vitamin B, 2 and folic acid levels, and thryroid-stimulating hormone levels. In this study, the first set of tests used for diagnostic purposes included the Wechsler Adult Intelligence Scale Revised, Wechsler Memory Scale-Revised, Auditory verbal learning Test and Wide-Range Achievement test-III. A second set of tests, which were used for research purposes, included the Mini-Mental State Examination, dementia rating Scale, Free and Cued Selective Reminding test, Boston Naming Test, Controlled Oral Word Association Test and category fluency procedures.

Verbal Fluency, Verbal Memory And Logical Memory

According to one embodiment, administration of the first agent which is an antagonist of 11-HSD1, in combination with a second agent which comprises a diuretic (preferably an anti-kaliuretic-diuretic) to an individual is capable of improving any one or more of his verbal fluency, his verbal memory, and his logical memory. The individual may be one who is suffering from Type 2 diabetes, or otherwise.

Verbal fluency, verbal memory and logical memory may be assessed by various tests as known in the art. Suitable tests include for example, those shown in Table 1 below.

TABLE 1
Tests for verbal fluency, verbal memory and logical memory.

| Test | Notes | Reference |
|---|---|---|
| California Verbal Learning | Examines several aspects of verbal | Construct validation |
| Test | learning, organization, and | of the California |
| | memory. Forms for adults and | Verbal Learning Test. |
| | children. | Delis DC, Freeland J, |
| | | Kramer JH, Kaplan |
| | | E. J Consult Clin |
| | | Psychol 1988 |
| | | Feb; 56(1): 123-30 |
| Controlled Oral Word | Different forms of this procedure | Borkowski JG, |
| Association Test | exist. Most frequently used for | Benton AL. Word |
| | assessing verbal fluency and the | fluency and brain |
| | ease with which a person can think | damage. |
| | of words that begin with a specific | Neuropsychologia |
| | letter. | 1967; 5: 135-140 |
| Delis-Kaplan Executive | Assesses key areas of executive | Delis D, Kaplan D |
| Function System | function (problem-solving, | Delis-Kaplan |
| | thinking flexibility, fluency, | Executive Function |
| | planning, deductive reasoning) in | System, |
| | both spatial and verbal modalities, | Psychological |
| | normed for ages 8-89. | Corporaton 2001 |
| Kaplan Baycrest | Assesses cognitive abilities in | Leach L, E Kaplan |
| Neurocognitive Assessment | adults, including attention, | Kaplan Baycrest |
| | memory, verbal fluency, spatial | Neurocognitive |
| | processing, and | Assessment, |
| | reasoning/conceptual shifting. | Psychological |
| | | Corporaion 2000 |
| Rey Auditory Verbal | This procedure evaluates the | Rey A. L'examen |
| Learning Test | ability to learn word lists. It is the | clinique en |
| | forerunner of other tests of verbal | psychologie. Paris: |
| | learning using lists of words. | Presses Universaires |
| | | de France, 1964 |
| Verbal (Word) Fluency | There are a variety of verbal | Borkowski JG, |
| Tests (various) | fluency tests in use. Each is | Benton AL. Word |
| | designed to measure the speed and | fluency and brain |
| | flexibility of verbal thought | damage. |
| | processes. (e.g., Controlled Oral | Neuropsychologia |
| | Word Association Test; Thurstone | 1967; 5: 135-140 |
| | Verbal Fluency) | |
| Wechsler Memory Scale | Standardised battery of tests- | Wechsler, D. (1945) |
| | provides overall review of all | A standardised |
| | major aspects of the memory | memory scale for |
| | system | clinical use. Journal |
| | | of Psychology, 1945, |
| | | 19, 87-95 |

In preferred embodiments, verbal fluency is assessed by a Controlled Word Association Test, and verbal memory is assessed by Rey Auditory Verbal Learning Test. In preferred embodiments, logical memory is assessed by a Wechsler Memory Scale.

Controlled Word Association Test

In a preferred embodiment, verbal fluency is assessed by a Controlled Word Association Test. The Controlled Word Association Test is sometimes known as the “Controlled Oral Word Association Test”, or COWAT or the Verbal Fluency Test, and these terms are used interchangeably in this document.

In such a test, a patient produces as many words as possible in 1 min. (each) for a specific letter (C, F, L). The tester asks the subject to say all the words that he can, that begin with the letter of the given alphabet, excluding their own names and numbers. The score is the sum of all the exact words produced from the subject in the three tasks in the time of a minute. Corrections to the score may be made for the age, sex and education of the subject. The number of errors (e.g., own names) and the repetitions are also estimated, and accounted for. The procedure typically takes 5 min to complete, and is designed to test language & executive/frontal skills.

The Controlled Word Association Test is described in Lezak, 1976 (LEZAK, M. Neuropsychological Assessment. New York: Oxford University Press, 1976).

Preferably, the methods and compositions described here enable an increase in verbal fluency as measured by a percentage change in the relevant cognitive score, i.e., a score obtained preferably by a Controlled Word Association Test.

Preferably, verbal fluency as assessed by a such a test is increased by 1%, 2%, 3%, 4%, 5%, 6%, 7%, 8%, 9%, 10%, 15%, 20%, 25%, 30%, 40%, 50%, 60%, 70%, 80%, 90%, 100% or more, by use of the methods and compositions described here. The percentage increase in performance or change in cognitive score may be identified in the average score of an individual or group of individuals administered with the methods and compositions described here, compared to the average score of an individual or group of individuals who have not been so administered. Preferably, the comparison is between an individual before and after administration.

A percentage increase in cognitive score may particularly be assessed as (score in COWAT with composition)−(score in COWAT with placebo)/(score in COWAT with placebo)×100%. In preferred embodiments, the percentage increase is assessed as a (score in COWAT with first agent and second agent)−(score in COWAT with placebo and second agent)/(score in COWAT with placebo and second agent)×100%. In particular, percentage increase may be assessed as a (score in COWAT with carbenoxolone and amiloride)−(score in COWAT with placebo and amiloride)/(score in COWAT with placebo and amiloride)×100%.

In preferred embodiments, the percentage increase in verbal fluency is at least 5%, more preferably at least 10%, more preferably 11% or thereabouts, or more.

Rey Auditory Verbal Learning Test

In a preferred embodiment, verbal memory is assessed by a Rey Auditory Verbal Learning Test (RAVLT or AVLT). Verbal memory is also known as “auditory memory”, and the two terms are used interchangeably in this document.

The Rey Auditory Verbal Learning Test is described in detail in Rey A. L'examen clinique en psychologie 1964. Paris:Presses Universaires, as well as in Michael Schmidt, Rey Auditory Verbal Learning Test: A Handbook (RAVLT), Psychological Assessment Resources, Inc., 16204 N. Florida Avenue, Lutz, Fla. 33549.

The Rey Auditory Verbal Learning Test enables the evaluation of verbal learning and memory for ages 7-89 years. The Rey Auditory Verbal Learning Test has evolved over the years, and several variations of the test have emerged; however, any of the different variations may be used to assess improvement in verbal memory according to the methods and compositions described here. A particularly preferred version of the test is the standard format set out below.

The standard Rey Auditory Verbal Learning Test format starts with a list of 15 words, which the examiner reads aloud at the rate of one word per second. The test-taker's task is to repeat all the words he or she can remember, in any order. This procedure is carried out a total of five times. Then, the examiner presents a second list of 15 words, allowing the test-taker only one attempt at recall. Immediately following this, the individual is asked to remember as many words as possible from the first list.

Thus, the test consists of five presentations with recall of a 15 word list, one presentation of a second 15-word list, and a sixth recall trial. This measures immediate memory span. Retention may be examined after 30 minutes or hours or days later.

The Rey Auditory Verbal Learning Test is useful in evaluating verbal learning and memory, including proactive inhibition, retroactive inhibition, retention, encoding versus retrieval, and subjective organization. Because the test is brief, straightforward, easy to understand, and appropriate for children, adolescents, and adults, it has gained widespread acceptance.

Preferably, the methods and compositions described here enable an increase in verbal memory as measured by a percentage change in the relevant cognitive score, i.e., a score obtained preferably by a Rey Auditory Verbal Learning Test.

Preferably, verbal memory as assessed by a such a test is increased by 1%, 2%, 3%, 4%, 5%, 6%, 7%, 8%, 9%, 10%, 15%, 20%, 25%, 30%, 40%, 50%, 60%, 70%, 80%, 90%, 100% or more, by use of the methods and compositions described here. The percentage increase in performance or change in cognitive score may be identified in the average score of an individual or group of individuals administered with the methods and compositions described here, compared to the average score of an individual or group of individuals who have not been so administered. Preferably, the comparison is between an individual before and after administration.

A percentage increase in cognitive score may particularly be assessed as (score in RAVLT with composition)−(score in RAVLT with placebo)/(score in RAVLT with placebo)×100%. In preferred embodiments, the percentage increase is assessed as a (score in RAVLT with first agent and second agent)−(score in RAVLT with placebo and second agent)/(score in RAVLT with placebo and second agent)×100%. In particular, percentage increase may be assessed as a (score in RAVLT with carbenoxolone and amiloride)−(score in RAVLT with placebo and amiloride)/(score in RAVLT with placebo and amiloride)×100%.

In such embodiments, the percentage increase in verbal memory is preferably at least 5%, preferably at least 7%, preferably at least 10%.

Logical Memory

Preferably, the methods and compositions described here enable an increase in logical memory as measured by a percentage change in the relevant cognitive score, i.e., a score obtained preferably by a Wechsler Memory Scale.

Preferably, logical memory as assessed by a such a test is increased by 1%, 2%, 3%, 4%, 5%, 6%, 7%, 8%, 9%, 10%, 15%, 20%, 25%, 30%, 40%, 50%, 60%, 70%, 80%, 90%, 100% or more, by use of the methods and compositions described here. The percentage increase in performance or change in cognitive score may be identified in the average score of an individual or group of individuals administered with the methods and compositions described here, compared to the average score of an individual or group of individuals who have not been so administered. Preferably, the comparison is between an individual before and after administration.

A percentage increase in cognitive score may particularly be assessed as (score in Wechsler Memory Scale with composition)-(score in Wechsler Memory Scale with placebo)/(score in Wechsler Memory Scale with placebo)×100%. In preferred embodiments, the percentage increase is assessed as a (score in Wechsler Memory Scale with first agent and second agent)−(score in Wechsler Memory Scale with placebo and second agent)/(score in Wechsler Memory Scale with placebo and second agent)×100%. In particular, percentage increase may be assessed as a (score in Wechsler Memory Scale with carbenoxolone and amiloride)−(score in Wechsler Memory Scale with placebo and amiloride)/(score in Wechsler Memory Scale with placebo and amiloride)×100%.

The methods of assessing increase in cognitive score mentioned above for either cognitive function may be applied to an individual. Thus, an increase may be observed in the cognitive score calculated as above in respect of a particular individual. Furthermore, the methods of assessing cognitive score increase may be applied to a group or a population; where this is the case, an increase may be observed as a positive change in mean cognitive score for a group or population, or it may be identified as a positive value of the mean of the change in cognitive score of all individuals in a group or population.

Agonists and Antagonists

The methods and compositions described here rely, in some embodiments, on blocking enzymatic or protein activity.

Agents which are capable of increasing the activity of an enzyme or protein are referred to as agonists of that activity. Similarly, antagonists reduce the activity of the enzyme or protein, i.e., an inhibitor. Thus, the first agent in the methods and compositions described here is an antagonist of 11β—HSD1, i.e., it is capable of decreasing or reducing the activity of 11β-HSD1. Furthermore, in some embodiments, the second agent is one which is capable of modulating an interaction between the first agent and 11β-HSD2, preferably capable of down-regulating an antagonistic effect of the first agent on 11β-HSD2.

Furthermore, the second agent may prevent the adverse consequences of a reduction in 11β-HSD2 activity, for example by acting as an antagonist of mineralocorticoid receptors or of mineralocorticoid receptor-regulated target gene products, such as the amiloride sensitive sodium channel or Na-K-ATPase.

The term “antagonist”, as used in the art, is generally taken to refer to a compound which binds to an enzyme and inhibits the activity of the enzyme. The term as used here, however, is intended to refer broadly to any agent which inhibits the activity of a molecule, not necessarily by binding to it. Accordingly, it includes agents which affect the expression of a protein such as a 11β-HSD1, or the biosynthesis of a molecule such as a 11β-HSD1, or the expression of modulators of the activity of 11β-HSD1. The specific activity which is inhibited may be any activity which is characteristic of the enzyme or molecule, for example, a dehydrogenase activity of 11β-HSD1. Assays for such activies are known in the art.

The antagonist may bind to and compete for one or more sites on the relevant molecule, for example, a 11β-HSD1 molecule, to reduce one or more of its activities (including a dehydrogenase activity). Preferably, such binding blocks the interaction between the molecule and another entity. However, the antagonist need not necessarily bind directly to a catalytic site, and may bind for example to an adjacent site, another protein (for example, a protein which is complexed with the enzyme) or other entity on or in the cell, so long as its binding reduces the activity of the enzyme or molecule.

Where antagonists of a enzyme such as 11β-HSD1 are concerned, an antagonist may include a substrate of the enzyme, or a fragment of this which is capable of binding to the enzyme. In addition, whole or fragments of a substrate generated natively or by peptide synthesis may be used to compete with the substrate for binding sites on the enzyme. Alternatively, or in addition, an immunoglobulin (for example, a monoclonal or polyclonal antibody) capable of binding to the enzyme may be used. The antagonist may also include a peptide or other small molecule which is capable of interfering with the binding interaction. Other examples of antagonists are set forth in greater detail below, and will also be apparent to the skilled person.

Blocking the activity of a enzyme such as an 11β-HSD1 may also be achieved by reducing the level of expression of the enzyme in the cell. For example, the cell may be treated with antisense compounds, for example oligonucleotides having sequences specific to 11β-HSD1mRNA.

As used herein, in general, the term “antagonist” includes but is not limited to agents such as an atom or molecule, wherein a molecule may be inorganic or organic, a biological effector molecule and/or a nucleic acid encoding an agent such as a biological effector molecule, a protein, a polypeptide, a peptide, a nucleic acid, a peptide nucleic acid (PNA), a virus, a virus-like particle, a nucleotide, a ribonucleotide, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid, a fatty acid and a carbohydrate. An agent may be in solution or in suspension (e.g., in crystalline, colloidal or other particulate form). The agent may be in the form of a monomer, dimer, oligomer, etc, or otherwise in a complex.

The terms “antagonist” and “agent” are also intended to include, a protein, polypeptide or peptide including, but not limited to, a structural protein, an enzyme, a cytokine (such as an interferon and/or an interleukin) an antibiotic, a polyclonal or monoclonal antibody, or an effective part thereof, such as an Fv fragment, which antibody or part thereof may be natural, synthetic or humanised, a peptide hormone, a receptor, a signalling molecule or other protein; a nucleic acid, as defined below, including, but not limited to, an oligonucleotide or modified oligonucleotide, an antisense oligonucleotide or modified antisense oligonucleotide, cDNA, genomic DNA, an artificial or natural chromosome (e.g. a yeast artificial chromosome) or a part thereof, RNA, including mRNA, tRNA, rRNA or a ribozyme, or a peptide nucleic acid (PNA); a virus or virus-like particles; a nucleotide or ribonucleotide or synthetic analogue thereof, which may be modified or unmodified; an amino acid or analogue thereof, which may be modified or unmodified; a non-peptide (e.g., steroid) hormone; a proteoglycan; a lipid; or a carbohydrate. Small molecules, including inorganic and organic chemicals, which bind to and occupy the active site of the polypeptide thereby making the catalytic site inaccessible to substrate such that normal biological activity is prevented, are also included. Examples of small molecules include but are not limited to small peptides or peptide-like molecules.

The antagonist or agent may comprise a protease which cleaves the relevant enzyme. Examples of proteases include aminopeptidase M, carboxypeptidase P, carboxypeptidase Y, caspase 1,4,5, caspase 2,3,7, caspase 6,8,9, chymotrypsin, Factor Xa, pepsin, TEV, thrombin, trypsin etc.

Modulators of 11β-HSD1 Activity

Agents which are capable of modulating 11β-HSD1 activity, including agents which are antagonists of 11β-HSD1, are well known in the art.

Monder C, White PC. “11β-Hydroxysteroid dehydrogenase.” Vitamins and Hormones 1993; 47: 187-271, provides an extensive list of such inhibitors in 1993. That list, given as Table IV therein, is incorporated herein by reference. Especially preferred are inhibitors of the reductase activity of 1 p-HSD1, which include 11-oxoprogesterone, 3α,17,21-trihydoxy-5β-pregnan-3-one, 21-hydroxy-pregn-4-ene-3,11,20-trione, androst-4-ene-3,11,20-trione and 3β-hydroxyandrost-5-en-17-one.

Carbenoxolone

In a particularly preferred embodiment, the first agent comprises an antagonist of 11β-HSD1 which is carbenoxolone.

Carbenoxolone is preferably administered at a rate of at or about 100 mg every 8 hours, preferably over a course of 4 weeks or more.

Preferably, carbenoxolone is administered to an individual at or about 1, 1.5, 2, 2.5, 3, 3.5, 4, 4.5, 5, 5.5, 6, 6.5, 7, 7.5, 8, 8.5, 9, 9.5 or up to 10 mg/kg/day of carbenoxolone. In a highly preferred embodiment, carbenoxolone is administered to an individual at a rate of about 4.5 mg/kg/day.

Carbenoxolone, as well as other antagonists and inhibitors of 11-βHSD1, and modes of administration thereof, are described for example from Walker et al., “Carbenoxolone Increases Hepatic Insulin Sensitivity in Man: A Novel Role for 11-oxosteroid Reductase in Enhancing Glucocorticoid Receptor Activation,” J. Clin. Endocrinology and Metabolism 80 (11): 3155-59 (1995); Gomez-Sanchez et al., “Central hypertensinogenic effects of glycyrrhizic acid and carbenoxolone,” Am J Physiol 263 (6 Pt 1): E1125-E1130 (1992) which showed that liquorice, glycyrrhizic acid, and carbenoxolone were known inhibitors, as well as the infusion of glycyrrhizic acid and carbenoxolone into the lateral ventricle of the brain of the rat at doses less than that which has an effect when infused subcutaneously, produces hypertension, showing that such compounds were administered subcutaneously, orally, and by infusion; see also Whorwood et al., “Liquorice inhibits 11 beta-hydroxysteroid dehydrogenase messenger ribonucleic acid levels and potentiates glucocorticoid hormone action,” Endocrinology 132 (6): 2287-92 (1993). Even further still, Homma et al., “A Novel 11 P-Hydroxsteroid Dehydrogenase Inhibitor Contained in Saiboku-To, a Herbal Remedy for Steroid-dependent Bronchial Asthma,” J. Pharm Pharmacol 46:305-309 (1994), Zhang et al., “Inhibition of 11β-Hydroxysteroid Dehydrogenase Obtained from Guinea Pig Kidney by Furosemide, Naringenin and Some Other Compounds,” J Steroid Biochem Molec Biol 49(1):81-85 (1994), and Lee et al., “Grapefruit juice and its flavenoids inhibit 11β-hydroxysteroid dehydrogenase,” Clin Pharmacol Ther 59:62-71 (1996), describe even more inhibitors that can be administered in known ways (both in terms of doses and routes of administration), such as flavenoids, which “are sold in tablet form in health food stores and drug stores,” and herbs or constituents of herbs. Moreover, Morris et al., “Endogenous 11 beta-hydroxysteroid dehydrogenase inhibitors and their role in glucocorticoid Na+ retention and hypertension,” Endocr Res 22(4):793-801 (1996) describe progesterone metabolites as 11β-HSD1 inhibitors, and progesterone is also a substance that can be administered, both in terms of doses and routes of administration, without difficulty by one skilled in the art.

Antibody Modulators of 11β-HSD1 Activity

The first agent which is an antagonist of 11β-HSD1 may comprise an antibody.

Antibodies, as used herein, refers to complete antibodies or antibody fragments capable of binding to a selected target, and including Fv, ScFv, Fab′ and F(ab′)₂, monoclonal and polyclonal antibodies, engineered antibodies including chimeric, CDR-grafted and humanised antibodies, and artificially selected antibodies produced using phage display or alternative techniques. Small fragments, such Fv and ScFv, possess advantageous properties for diagnostic and therapeutic applications on account of their small size and consequent superior tissue distribution.

Antibody antagonists of 11βHSD1 may be obtained from animal serum, or, in the case of monoclonal antibodies or fragments thereof, produced in cell culture. Recombinant DNA technology may be used to produce the antibodies according to established procedure, in bacterial or preferably mammalian cell culture. The selected cell culture system preferably secretes the antibody product.

Therefore, we describe a process for the production of an antibody antagonist of 11βHSD1 comprising culturing a host, e.g. E. coli or a mammalian cell, which has been transformed with a hybrid vector comprising an expression cassette comprising a promoter operably linked to a first DNA sequence encoding a signal peptide linked in the proper reading frame to a second DNA sequence encoding said protein, and isolating said protein.

Multiplication of hybridoma cells or mammalian host cells in vitro is carried out in suitable culture media, which are the customary standard culture media, for example Dulbecco's Modified Eagle Medium (DMEM) or RPMI 1640 medium, optionally replenished by a mammalian serum, e.g. foetal calf serum, or trace elements and growth sustaining supplements, e.g. feeder cells such as normal mouse peritoneal exudate cells, spleen cells, bone marrow macrophages, 2-aminoethanol, insulin, transferrin, low density lipoprotein, oleic acid, or the like. Multiplication of host cells which are bacterial cells or yeast cells is likewise carried out in suitable culture media known in the art, for example for bacteria in medium LB, NZCYM, NZYM, NZM, Terrific Broth, SOB, SOC, 2×YT, or M9 Minimal Medium, and for yeast in medium YPD, YEPD, Minimal Medium, or Complete Minimal Dropout Medium.

In vitro production provides relatively pure antibody preparations and allows scale-up to give large amounts of the desired antibodies. Techniques for bacterial cell, yeast or mammalian cell cultivation are known in the art and include homogeneous suspension culture, e.g. in an airlift reactor or in a continuous stirrer reactor, or immobilised or entrapped cell culture, e.g. in hollow fibres, microcapsules, on agarose microbeads or ceramic cartridges.

Large quantities of the desired antibodies can also be obtained by multiplying mammalian cells in vivo. For this purpose, hybridoma cells producing the desired antibodies are injected into histocompatible mammals to cause growth of antibody-producing tumours. Optionally, the animals are primed with a hydrocarbon, especially mineral oils such as pristane (tetramethyl-pentadecane), prior to the injection. After one to three weeks, the antibodies are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pre-treated with pristane, and, after one to two weeks, ascitic fluid is taken from the animals.

The foregoing, and other, techniques are discussed in, for example, Kohler and Milstein, (1975) Nature 256:495-497; U.S. Pat. No. 4,376,110; Harlow and Lane, Antibodies: a Laboratory Manual, (1988) Cold Spring Harbor, incorporated herein by reference. Techniques for the preparation of recombinant antibody molecules is described in the above references and also in, for example, EP 0623679; EP 0368684 and EP 0436597, which are incorporated herein by reference.

The cell culture supernatants are screened for the desired antibodies, preferentially by immunofluorescent staining of cells expressing the desired antigen by immunoblotting, by an enzyme immunoassay, e.g. a sandwich assay or a dot-assay, or a radioimmunoassay.

For isolation of the antibodies, the immunoglobulins in the culture supernatants or in the ascitic fluid may be concentrated, e.g. by precipitation with ammonium sulphate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or (immuno-)affinity chromatography, e.g. affinity chromatography with an 11β-HSD1 molecule or with Protein-A.

Recombinant DNA technology may be used to improve the antibody antagonists of 11βHSD1. Thus, chimeric antibodies may be constructed in order to decrease the immunogenicity thereof in diagnostic or therapeutic applications. Moreover, immunogenicity may be minimised by humanising the antibodies by CDR grafting [see European Patent 0 239 400 (Winter)] and, optionally, framework modification [European Patent 0 239 400; reviewed in international patent application WO 90/07861 (Protein Design Labs)].

We also describe hybridoma cells secreting monoclonal antibody antagonists of 11βHSD1. Preferred hybridoma cells are genetically stable, secrete monoclonal antibody antagonists of 11βHSD1 of the desired specificity and can be activated from deep-frozen cultures by thawing and recloning.

We describe a process for the preparation of a hybridoma cell line secreting monoclonal antibodies directed to a 11β-HSD1 molecule, characterised in that a suitable mammal, for example a Balb/c mouse, is immunised with a purified 11β-HSD1 molecule, an antigenic carrier containing a purified 11β-HSD1 molecule or with cells bearing 11β-HSD1, antibody-producing cells of the immunised mammal are fused with cells of a suitable myeloma cell line, the hybrid cells obtained in the fusion are cloned, and cell clones secreting the desired antibodies are selected. For example spleen cells of Balb/c mice immunised with cells bearing 11-HSD1 are fused with cells of the myeloma cell line PAI or the myeloma cell line Sp2/0-Ag14, the obtained hybrid cells are screened for secretion of the desired antibodies, and positive hybridoma cells are cloned.

Preferred is a process for the preparation of a hybridoma cell line, characterised in that Balb/c mice are immunised by injecting subcutaneously and/or intraperitoneally between 10 and 107 and 108 cells of human tumour origin which express 11β-HSD1 containing a suitable adjuvant several times, e.g. four to six times, over several months, e.g. between two and four months, and spleen cells from the immunised mice are taken two to four days after the last injection and fused with cells of the myeloma cell line PAI in the presence of a fusion promoter, preferably polyethylene glycol. Preferably the myeloma cells are fused with a three- to twentyfold excess of spleen cells from the immunised mice in a solution containing about 30% to about 50% polyethylene glycol of a molecular weight around 4000. After the fusion the cells are expanded in suitable culture media as described hereinbefore, supplemented with a selection medium, for example HAT medium, at regular intervals.

We also provide intracellular antibodies, capable of operating within a cell, for the regulation of 11β-HSD1 levels intracellularly. Intracellular antibodies are advantageously scFv antibodies, expressed intracellularly from expression vectors as is known in the art.

Intracellular antibodies or intrabodies have been demonstrated to function in antigen recognition in the cells of higher organisms (reviewed in Cattaneo, A. & Biocca, S. (1997) Intracellular Antibodies: Development and Applications. Landes and Springer-Verlag). This interaction can influence the function of cellular proteins which have been successfully inhibited in the cytoplasm, the nucleus or in the secretory pathway. This efficacy has been demonstrated for viral resistance in plant biotechnology (Tavladoraki, P., et al. (1993) Nature 366: 469-472) and several applications have been reported of intracellular antibodies binding to HIV viral proteins (Mhashilkar, A. M., et al. (1995) EMBO J 14: 1542-51; Duan, L. & Pomerantz, R. J. (1994) Nucleic Acids Res 22: 5433-8; Maciejewski, J. P., et al. (1995) Nat Med 1: 667-73; Levy-Mintz, P., et al. (1996) J. Virol. 70: 8821-8832) and to oncogene products (Biocca, S., Pierandrei-Amaldi, P. & Cattaneo, A. (1993) Biochem Biophys Res Commun 197: 422-7; Biocca, S., Pierandrei-Amaldi, P., Campioni, N. & Cattaneo, A. (1994) Biotechnology (N Y) 12: 396-9; Cochet, O., et al. (1998) Cancer Res 58: 1170-6).

Anti-Kaliuretic Diuretics

According to the methods and compositions described here, the second agent may comprise a diuretic. Preferably, the diuretic is such that it selectively enhances the excretion of sodium ions without causing an increase in excretion of potassium ions.

Thus, in a preferred embodiment, the second agent comprises an antikaliuretic-diuretic agent. Antikaliuretic-diuretics, also known as “potassium-sparing diuretics”, comprise a class of drugs capable of blocking the exchange of sodium for potassium and hydrogen ions in the distal tubule, causing an increase in the excretion of sodium and chloride with a negligible increase in potassium excretion.

In highly preferred embodiments, the second agent is capable of modulating the inhibition of the first agent on 11β-HSD1, preferably down-regulating, most preferably reversing such inhibition. Preferably, the second agent is not capable of binding to mineralocorticoid receptors; more preferably the second agent is not capable of blocking mineralocorticoid receptors.

The second agent may be any molecule or atom which has a potassium sparing diuretic effect. Examples of such agents are known in the art, and include those described in further detail below. Further examples of potassium sparing diuretics which may be used as second agents in the methods and compositions described here include Triamterine (also known as Triamterene), Trimethoprim and Tetroxoprim (Potassium-sparing renal effects of trimethoprim and structural analogues. Gabriels G, Stockem E, Greven J Nephron 86: 70-78 2000), as well as leptin (Human leptin has natriuretic activity in the rat. Jackson E K, Li P. American Journal of Physiology-Renal Physiology 41: F333-F338 1997.) The second agent may alternatively or in addition comprise triamterene or potassium canrenoate.

Amiloride

Amiloride is disclosed in U.S. Pat. No. 3,313,813 to E. Cragoe. Salts, esters and derivatives of amiloride, which may also be used in the methods and compositions described here, are described in U.S. Pat. No. 5,260,091.

Preferably, the second agent comprises Amiloride HCl. Amiloride HCl, an antikaliuretic-diuretic agent, is a pyrazine-carbonyl-guanidine that is unrelated chemically to other known antikaliuretic or diuretic agents. It is the salt of a moderately strong base (pKa 8.7). It is designated chemically as 3,5-diamino-6-chloro-N-(diaminomethylene) pyrazinecarboxamide monohydrochloride, dihydrate and has a molecular weight of 302.12. Its empirical formula is C₆H₈ClN₇O.HCl.2H₂O.

MIDAMOR (Amiloride HCl) is available for oral use as tablets containing 5 mg of anhydrous amiloride HCl. Each tablet contains the following inactive ingredients: calcium phosphate, D&C Yellow 10, iron oxide, lactose, magnesium stearate and starch.

Other forms of amiloride may be used in the methods and compositions described here. For example, Amiloride-thiazide is described in U.S. Pat. No. 4,898,729. Amiloride compositions may be prepared by the procedures disclosed in U.S. Pat. No. 3,313,813. The synthesis and uses of a salt of amiloride, amiloride citrate is described in U.S. Pat. No. 4,190,655. This patent also describes certain pharmaceutical compositions, which may be used in the methods and compositions described here.

Preferably, amiloride is administered to an individual at a rate of about 10 mg per day, preferably over a course of 4 weeks or more.

Preferably, amiloride is administered to an individual at or about 0.1, 0.2, 0.3, 0.4, 0.5, 0.6, 0.7, 0.8, 0.9, or up to 1 mg/kg/day of amiloride. In a highly preferred embodiment, amiloride is administered to an individual at a rate of about 0.15 mg/kg/day.

Aldosterone Antagonists

In some embodiments of the methods and compositions described here, the second agent comprises an aldosterone antagonist (also known as an anti-aldosterone drug, or a aldosterene blocking agent). A preferred aldosterone antagonist suitable for use in the methods and compositions described here is spironolactone, Eplerenone, or potassium canrenoate.

Preferred antagonists of aldosterone are those which occupy aldosterone receptor sites without triggering the normal receptor activity. This competitive binding reaction reduces the ability of aldosterone molecules to bind to and trigger activity at such receptors. As used herein, “aldosterone antagonist” refers to a compound that suppresses the receptor-mediated activity of aldosterone, as well as compounds which reduce the amount of aldosterone synthesised or secreted by the adrenal cortex, such as mespirenone. However, in a preferred embodiment, aldosterone antagonists are those which suppress the receptor mediated activity of aldosterone.

The second agent may in general comprise a mineralocorticoid receptor antagonist.

Spirolactones

In one embodiment, the second agent comprises an aldosterone antagonist which is a spirolactone, preferably a spironolactone.

A number of drugs have been identified which can inhibit the activity of aldosterone in the body, including spirolactones. Accordingly, the second agent may suitably comprise a spirolactone. The term “spirolactone” indicates that a lactone ring (i.e., a cyclic ester) is attached to another ring structure in a spiro configuration (i.e., the lactone ring shares a single carbon atom with the other ring). Spirolactones which are coupled to steroids are the most important class of spirolactones from a pharmaceutical perspective, so they are widely referred to in the pharmaceutical arts simply as spirolactones. As used herein, “spirolactone” refers to a molecule comprising a lactone structure coupled via a spiro configuration to a steroid structure or steroid derivative.

Spironolactone

One particular spirolactone which functions as an effective aldosterone antagonist is called spironolactone, and the methods and compositions described here in a highly preferred embodiment preferably employ spironolactone as a second agent.

Spironolactone is marketed as an anti-hypertensive and diuretic drug by G. D. Searle (Skokie, Ill.) under the trademarks “Aldactone” and “Aldactazide.” Spironolactone is the name commonly used by chemists; the full chemical name is 17-hydroxy-7-alpha-mercapto-3-oxo-17-alpha-pregn-4-ene-21-carboxylic acid gamma-lactone acetate. This compound, its activities, and modes of synthesis and purification are described in a number of U.S. Pat. Nos. including U.S. Pat. No. 3,013,012 (Cella and Tweit 1961) and U.S. Pat. No. 4,529,811 (Hill and Erickson 1985).

Spironolactone is known to have unwanted effects to reduce cognitive function (Yau J L W, Noble J and Seckl J R (1999). Neurosci Lett 277: 45-48). However, according to the methods and compositions described here, a combination of a first agent which is an antagonist of 11β-HSD1 together with a spironolactone is capable of improving cognitive abilities, including any one or more of verbal fluency, verbal memory and logical memory. Such a combination may therefore be used for improving cognitive ability and in treating Mild Cognitive Impairment.

When spironolactone is used to suppress aldosterone activity, it promotes the elimination of fluid and sodium by the body, primarily via the kidneys and its formation of urine. Both of these effects help control hypertension in people suffering from high blood pressure. Spironolactone is therefore used to treat hypertension due to excessive secretion of aldosterone. The minimum effective anti-hypertensive dosage in adults is about 50 milligrams (mg) per day; dosages often exceed this, and dosages of 200 to 400 mg/day are common for chronic treatment. Since spironolactone is metabolized and secreted fairly rapidly, typical administration involves pills containing 25 to 100 mg, taken four times daily. Such dosages may be used as a guideline for the administration of spironolactone according to the methods and compositions described here.

Spironolactone is a synthetic steroid with an aldosterone-like structure, and acts as a competitive antagonist at aldosterone receptors. The most important of these receptors are situated in the distal portion of the renal tubules. Spironolactone thus inhibits sodium and water reabsorption while sparing the potassium and magnesium metabolism. Spironolactone is also an anti-androgen.

Thus, primary effects of spironolactone include any or all of the following: competitive antagonism of aldosterone by competitive binding to mineralocorticoid receptors; inhibition of reabsorption of sodium and reduction in the elimination of potassium, H+ ions and calcium; stimulation of system renin angiotensin aldosterone, related to the sodium depletion; increase synthesis of E2 prostaglandin and reduction of formation of thromboxane A2 (Prost Leuko Med 1986;24: 103-109). Action on the distal tubule depends on the blood concentration of aldosterone and the sodium concentration on the level of the distal tubule. Secondary effects of spironolactone include any or all of the following: androgenic anti action by blocking of the synthesis of the 17 Oh-testosterone, and increase in the progesterone rate; probable action on the cells of Leydig and those of the suprarenal cortex (J Urology 1978; 119:375); and induction of enzyme expression by hepatic microsomes.

Examples of formulations which contain spironolactone include ALDACTONE® 100 TABLETS, ALDACTONE® 25 TABLETS, ALDAZIDE® TABLETS, ROLAB-SPIRONOLACTONE 25 Tablets, SPIRACTIN 100 TABLETS, SPIRACTIN® TABLETS,® and TENSIN TABLETS (Searle).

Sprionolactone, and its uses, are described in various documents, including U.S. Pat. Nos. 6,150,347, 6,093,708, 5,668,125, 5,668,124, 5,529,992 and 5,506,222. A “lipid profile” is the level of lipids present in the blood. A lipid profile usually includes the total cholesterol, high density lipoprotein (HDL) cholesterol, triglycerides, and the calculated low density lipoprotein (LDL) cholesterol. A lipid profile comprises at least the level of one or more triglycerides and the level of HDL cholesterol.

Eplerenone

In other embodiments, the second agent comprises an aldosterone antagonist, preferably a Selective Aldosterone Receptor Antagonist (SARA). Preferably, the second agent comprises Eplerenone (Pharmacia).

Eplerenone comprises a 9,11-epoxy steroids; the full chemical name of Eplerenone is methyl hydrogen 9,11.alpha.-epoxy-17.alpha.-hydroxy-3-oxopregn-4-ene-7.alpha.,21-dicarboxy late, .gamma.-lactone. Eplerenone is also known as epoxymexrenone. Methods for the synthesis of Eplerenone are described in U.S. Pat. Nos. 4,559,332, 6,335,441, 6,331,622, 6,258,946, 6,180,780 and 5,981,744. These documents also describe a number of compounds related to Eplerenone, which are suitable for use in the methods and compositions described here.

Eplerenone, as well as its effects, is described in John A. Delyani, Ricardo Rocha, Chyung S. Cook, Dwain S. Tolbert, Stuart Levin, Barbara Roniker, Diane L. Workman, Yuen-lung L. Sing, Brian Whelihan (2001), Eplerenone: A Selective Aldosterone Receptor Antagonist (SARA), Cardiovascular Drug Reviews, Vol. 19, No. 3, pp. 185-200. Eplerenone is also described in Rajagopalan S, Duquaine D, Han Z, et al. Selective aldosterone receptor blockade improves endothelial function in diet induced atherosclerosis. Circulation. 2001;37:303A, Giles et al., American Journal Of Geriatric Cardiology 2001 VOL. 10 NO. 3, 66.

11-β Hydroxysteroid Dehydrogenase Type 1

11β-HSD1 is known in the art (A. K. Agarwal, C. Monder, B. Eckstein, and P. C. White. Cloning and expression of rat cDNA encoding corticosteroid 11β-dehydrogenase. J. Biol. Chem. 264:18939-18943, 1989) and is commonly expressed in white adipose tissue and liver.

The structure of 11β-HSD1 and the human gene encoding it are known (GenBank NM_{—}005525.1 GI:5031764). Human cDNA clones encoding 11β-hydroxysteroid dehydrogenase type I were isolated from a testis cDNA library by hybridisation with the previously isolated rat 11-HSD cDNA clone (Tannin, et al., J. Biol. Chem. 266: 16653-16658, 1991). The cDNA contained an open reading frame of 876 nucleotides, which predicted a protein of 292 amino acids. The sequence was 77% identical at the amino acid level to the rat 11-HSD. By hybridisation of the human cDNA to a human/hamster hybrid cell panel (72) localised the 11β-HSD1 gene to chromosome 1. The localisation was confirmed by isolating the gene from a chromosome 1-specific library using the cDNA as a probe. The gene consists of 6 exons and is at least 9 kb long.

The activities of 11-βHSD1, including its dehydrogenase activity, are known in the art, and assays to determine these activities are also known. Antagonists or inhibitors of 11-βHSD1 may be identified by contacting a candidate molecule with 11-βHSD1, and detecting the relevant activity in a suitable assay (e.g., detecting dehydrogenase activity in a standard dehydrogenase activity assay).

Agents Which Modulate 11β-HSD1 Expression

The modulation of gene expression is known to those skilled in the art to be achievable in a number of ways in vivo and in vitro. Antisense techniques as well as direct gene manipulation are known for use in modulating gene expression. We therefore disclose the use of antisense nucleic acids, which may incorporate natural or modified nucleotides, or both, ribozymes, including hammerhead ribozymes, gene knockout such as by homologous recombination, and other techniques for reducing gene expression levels of 11-βHSD1. In addition, we disclose certain inhibitors of 11-βHSD1 activity, as well as methods to determine compounds capable of use as such inhibitors.

Administration

The first and/or second agents, or a composition comprising them, may be delivered by conventional medicinal approaches, in the form of a pharmaceutical composition. A pharmaceutical composition in the context of the present document is a composition of matter comprising at least an inhibitor or antagonist of 11β-HSD1, together with a second agent which comprises a diuretic, preferably an anti-kaliuretic-diuretic, as an active ingredient.

Advantageously, the composition comprises a combination of an 11β-HSD1 inhibitor, together with a second agent which is capable of modulating an interaction between the first agent and 11β-HSD2. Preferably, the second agent is capable of down-regulating an antagonistic effect of the first agent on 11-βHSD2, or of preventing activation of mineralocorticoid receptors or their adverse effects.

The active ingredient(s) of a pharmaceutical composition is contemplated to exhibit excellent therapeutic activity, for example, in the alleviation of cardiovascular diseases. Dosage regimes may be adjusted to provide the optimum therapeutic response. For example, several divided doses may be administered daily or the dose may be proportionally reduced as indicated by the exigencies of the therapeutic situation.

The active compound may be administered in a convenient manner such as by the oral, intravenous (where water soluble), intramuscular, subcutaneous, intranasal, intradermal or suppository routes or implanting (e.g. using slow release molecules). Depending on the route of administration, the active ingredient may be required to be coated in a material to protect said ingredients from the action of enzymes, acids and other natural conditions which may inactivate said ingredient.

In order to administer the combination by other than parenteral administration, it will be coated by, or administered with, a material to prevent its inactivation. For example, the combination may be administered in an adjuvant, co-administered with enzyme inhibitors or in liposomes. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin.

Liposomes include water-in-oil-in-water CGF emulsions as well as conventional liposomes.

The active compound may also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene gloycol, and the like), suitable mixtures thereof, and vegetable oils. The proper fluidity can be maintained, for example, by the use of a coating such as lecithin, by the maintenance of the required particle size in the case of dispersion and by the use of superfactants.

The prevention of the action of microorganisms can be brought about by various antibacterial and antifungal agents, for example, parabens, chlorobutanol, phenyl, sorbic acid, thirmerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compound in the required amount in the appropriate solvent with various of the other ingredients enumerated above, as required, followed by filtered sterilisation. Generally, dispersions are prepared by incorporating the sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the combination of polypeptides is suitably protected as described above, it may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. The amount of active compound in such therapeutically useful compositions in such that a suitable dosage will be obtained.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms are dictated by and directly dependent on (a) the unique characteristics of the active material and the particular therapeutic effect to be achieved, and (b) the limitations inherent in the art of compounding such as active material for the treatment of disease in living subjects having a diseased condition in which bodily health is impaired.

The principal active ingredients are compounded for convenient and effective administration in effective amounts with a suitable pharmaceutically acceptable carrier in dosage unit form. In the case of compositions containing supplementary active ingredients, the dosages are determined by reference to the usual dose and manner of administration of the said ingredients.

In some embodiments, the first agent which is an antagonist of 11β-HSD1, and the second agent which comprises a diuretic or an anti-kaliuretic-diuretic (as described in detail elsewhere) may be provided in the form of a pharmaceutical composition.

While it is possible for the composition comprising the first and second agents to be administered alone, it is preferable to formulate the active ingredient or ingredients as a pharmaceutical formulation. We therefore also disclose pharmaceutical compositions comprising a first agent which is an antagonist of 11β-HSD1, together with a second agent which comprises a diuretic, preferably an anti-kaliuretic-diuretic. We also disclose a pharmaceutical composition comprising a first agent which is an antagonist of 11β-HSD1, suitable for administration in conjunction with a second agent which comprises a diuretic, preferably an anti-kaliuretic-diuretic. We furthermore disclose a pharmaceutical composition comprising a second agent which comprises a diuretic, preferably an anti-kaliuretic-diuretic, suitable for administration in conjunction with a first agent as described.

Such pharmaceutical compositions are useful for delivery of the first or second agents, or both, preferably in the form of a composition as described, to an individual for the treatment or alleviation of symptoms as described.

The composition may include the first agent which is an antagonist of 11-HSD1, optionally together with a second agent which comprises a diuretic, preferably an anti-kaliuretic-diuretic, or a fragment, homologue, variant or derivative thereof, a structurally related compound, or an acidic salt of either. The pharmaceutical formulations comprise an effective amount of the first and/or second agent, fragment, homologue, variant or derivative thereof, together with one or more pharmaceutically-acceptable carriers. An “effective amount” is the amount sufficient to alleviate at least one symptom of a disease as described, for example, mild cognitive impairment (MCI). Furthermore, where verbal fluency and/or verbal memory and/or logical memory are concerned, an “effective amount” is the amount sufficient to provide an improvement in verbal fluency or verbal memory or logical memory, as the case may be.

The effective amount will vary depending upon the particular disease or syndrome to be treated or alleviated, as well as other factors including the age and weight of the patient, how advanced the disease etc state is, the general health of the patient, the severity of the symptoms, and whether the first and/or second agent or variant or derivative thereof is being administered alone or in combination with other therapies.

Suitable pharmaceutically acceptable carriers are well known in the art and vary with the desired form and mode of administration of the pharmaceutical formulation. For example, they can include diluents or excipients such as fillers, binders, wetting agents, disintegrators, surface-active agents, lubricants and the like. Typically, the carrier is a solid, a liquid or a vaporizable carrier, or a combination thereof. Each carrier should be “acceptable” in the sense of being compatible with the other ingredients in the formulation and not injurious to the patient. The carrier should be biologically acceptable without eliciting an adverse reaction (e.g. immune response) when administered to the host.

The pharmaceutical compositions disclosed here include those suitable for topical and oral administration, with topical formulations being preferred where the tissue affected is primarily the skin or epidermis (for example, psoriasis, eczema and other epidermal diseases). The topical formulations include those pharmaceutical forms in which the composition is applied externally by direct contact with the skin surface to be treated. A conventional pharmaceutical form for topical application includes a soak, an ointment, a cream, a lotion, a paste, a gel, a stick, a spray, an aerosol, a bath oil, a solution and the like. Topical therapy is delivered by various vehicles, the choice of vehicle can be important and generally is related to whether an acute or chronic disease is to be treated. As an example, an acute skin proliferation disease generally is treated with aqueous drying preparations, whereas chronic skin proliferation disease is treated with hydrating preparations. Soaks are the easiest method of drying acute moist eruptions. Lotions (powder in water suspension) and solutions (medications dissolved in a solvent) are ideal for hairy and intertriginous areas. Ointments or water-in-oil emulsions, are the most effective hydrating agents, appropriate for dry scaly eruptions, but are greasy and depending upon the site of the lesion sometimes undesirable. As appropriate, they can be applied in combination with a bandage, particularly when it is desirable to increase penetration of the agent composition into a lesion. Creams or oil-in-water emulsions and gels are absorbable and are the most cosmetically acceptable to the patient. (Guzzo et al, in Goodman & Gilman's Pharmacological Basis of Therapeutics, 9th Ed., p. 1593-15950 (1996)). Cream formulations generally include components such as petroleum, lanolin, polyethylene glycols, mineral oil, glycerin, isopropyl palmitate, glyceryl stearate, cetearyl alcohol, tocopheryl acetate, isopropyl myristate, lanolin alcohol, simethicone, carbomen, methylchlorisothiazolinone, methylisothiazolinone, cyclomethicone and hydroxypropyl methylcellulose, as well as mixtures thereof.

Other formulations for topical application include shampoos, soaps, shake lotions, and the like, particularly those formulated to leave a residue on the underlying skin, such as the scalp (Arndt et al, in Dermatology In General Medicine 2:2838 (1993)).

In general, the concentration of the composition in the topical formulation is in an amount of about 0.5 to 50% by weight of the composition, preferably about 1 to 30%, more preferably about 2-20%, and most preferably about 5-10%. The concentration used can be in the upper portion of the range initially, as treatment continues, the concentration can be lowered or the application of the formulation may be less frequent. Topical applications are often applied twice daily. However, once-daily application of a larger dose or more frequent applications of a smaller dose may be effective. The stratum corneum may act as a reservoir and allow gradual penetration of a drug into the viable skin layers over a prolonged period of time.

In a topical application, a sufficient amount of active ingredient must penetrate a patient's skin in order to obtain a desired pharmacological effect. It is generally understood that the absorption of drug into the skin is a function of the nature of the drug, the behaviour of the vehicle, and the skin. Three major variables account for differences in the rate of absorption or flux of different topical drugs or the same drug in different vehicles; the concentration of drug in the vehicle, the partition coefficient of drug between the stratum corneum and the vehicle and the diffusion coefficient of drug in the stratum corneum. To be effective for treatment, a drug must cross the stratum corneum which is responsible for the barrier function of the skin. In general, a topical formulation which exerts a high in vitro skin penetration is effective in vivo. Ostrenga et al (J. Pharm. Sci., 60:1175-1179 (1971) demonstrated that in vivo efficacy of topically applied steroids was proportional to the steroid penetration rate into dermatomed human skin in vitro.

A skin penetration enhancer which is dermatologically acceptable and compatible with the agent can be incorporated into the formulation to increase the penetration of the active compound(s) from the skin surface into epidermal keratinocytes. A skin enhancer which increases the absorption of the active compound(s) into the skin reduces the amount of agent needed for an effective treatment and provides for a longer lasting effect of the formulation. Skin penetration enhancers are well known in the art. For example, dimethyl sulfoxide (U.S. Pat. No. 3,711,602); oleic acid, 1,2-butanediol surfactant (Cooper, J. Pharm. Sci., 73:1153-1156 (1984)); a combination of ethanol and oleic acid or oleyl alcohol (EP 267,617), 2-ethyl-1,3-hexanediol (WO 87/03490); decyl methyl sulphoxide and Azone.RTM. (Hadgraft, Eur. J. Drug. Metab. Pharmacokinet, 21:165-173 (1996)); alcohols, sulphoxides, fatty acids, esters, Azone.RTM., pyrrolidones, urea and polyoles (Kalbitz et al, Pharmazie, 51:619-637 (1996));

Terpenes such as 1,8-cineole, menthone, limonene and nerolidol (Yamane, J. Pharmacy & Pharmocology, 47:978-989 (1995)); Azone.RTM. and Transcutol (Harrison et al, Pharmaceutical Res. 13:542-546 (1996)); and oleic acid, polyethylene glycol and propylene glycol (Singh et al, Pharmazie, 51:741-744 (1996)) are known to improve skin penetration of an active ingredient.

Levels of penetration of an agent or composition can be determined by techniques known to those of skill in the art. For example, radiolabeling of the active compound, followed by measurement of the amount of radiolabeled compound absorbed by the skin enables one of skill in the art to determine levels of the composition absorbed using any of several methods of determining skin penetration of the test compound. Publications relating to skin penetration studies include Reinfenrath, W G and G S Hawkins. The Weaning Yorkshire Pig as an Animal Model for Measuring Percutaneous Penetration. In:Swine in Biomedical Research (M. E. Tumbleson, Ed.) Plenum, New York, 1986, and Hawkins, G. S. Methodology for the Execution of In Vitro Skin Penetration Determinations. In: Methods for Skin Absorption, B W Kemppainen and W G Reifenrath, Eds., CRC Press, Boca Raton, 1990, pp. 67-80; and W. G. Reifenrath, Cosmetics & Toiletries, 110:3-9 (1995).

For some applications, it is preferable to administer a long acting form of agent or composition using formulations known in the arts, such as polymers. The agent can be incorporated into a dermal patch (Junginger, H. E., in Acta Pharmaceutica Nordica 4:117 (1992); Thacharodi et al, in Biomaterials 16:145-148 (1995); Niedner R., in Hautarzt 39:761-766 (1988)) or a bandage according to methods known in the arts, to increase the efficiency of delivery of the drug to the areas to be treated.

Optionally, the topical formulations described here can have additional excipients for example; preservatives such as methylparaben, benzyl alcohol, sorbic acid or quaternary ammonium compound; stabilizers such as EDTA, antioxidants such as butylated hydroxytoluene or butylated hydroxanisole, and buffers such as citrate and phosphate.

The pharmaceutical composition can be administered in an oral formulation in the form of tablets, capsules or solutions. An effective amount of the oral formulation is administered to patients 1 to 3 times daily until the symptoms of the disease alleviated. The effective amount of agent depends on the age, weight and condition of a patient. In general, the daily oral dose of agent is less than 1200 mg, and more than 100 mg. The preferred daily oral dose is about 300-600 mg. Oral formulations are conveniently presented in a unit dosage form and may be prepared by any method known in the art of pharmacy. The composition may be formulated together with a suitable pharmaceutically acceptable carrier into any desired dosage form. Typical unit dosage forms include tablets, pills, powders, solutions, suspensions, emulsions, granules, capsules, suppositories. In general, the formulations are prepared by uniformly and intimately bringing into association the agent composition with liquid carriers or finely divided solid carriers or both, and as necessary, shaping the product. The active ingredient can be incorporated into a variety of basic materials in the form of a liquid, powder, tablets or capsules to give an effective amount of active ingredient to treat the disease.

Other therapeutic agents suitable for use herein are any compatible drugs that are effective for the intended purpose, or drugs that are complementary to the agent formulation. The formulation utilized in a combination therapy may be administered simultaneously, or sequentially with other treatment, such that a combined effect is achieved.

Metabolic Syndrome

The methods and compositions described here are suitable for the treatment of any of the symptoms of metabolic syndrome, as described below.

Metabolic syndrome is emerging as one of the major medical and public health problems both in the United States and worldwide. It is characterised by hypertension, hypertriglyceridaemia, and hyperglycaemia, is exacerbated by obesity, and constitutes a risk factor for coronary heart disease. The methods and compositions described here are suitable for promotion of an atheroprotective lipid profile in an individual.

Coronary heart disease is a condition that manifests as either heart attack (myocardial infarction), heart failure or chest pain (angina pectoris). It is caused by a narrowing and hardening of the coronary arteries (atherosclerosis). One of the primary features of atherosclerosis is the accumulation of cholesterol within the walls of the coronary arteries. Risk factors for coronary heart disease are the underlying causes of atherosclerosis. There are three major causes of coronary atherosclerosis: elevated LDL cholesterol, cigarette smoking, and the metabolic syndrome. Among these LDL cholesterol is the primary cause of atherosclerosis. When the blood level of LDL is increased, atherosclerosis is initiated and sustained. Cigarette smoking and the metabolic syndrome nevertheless constitute significant risk factors.

The metabolic syndrome is composed of individual risk factors that in aggregate greatly raise the risk for coronary heart disease. The metabolic risk factors that make up this syndrome are high triglycerides, small LDL particles, low HDL cholesterol, high blood pressure, high blood glucose, a tendency for blood clotting (thrombosis), and chronic inflammation. Taken in aggregate, these risk factors accelerate the development of atherosclerosis when they occur in the presence of elevated LDL cholesterol. When LDL-cholesterol levels are very low, the risk factors of the metabolic syndrome may have less effect on atherogenesis; but once LDL levels rise, these other risk factors are believed to become increasingly atherogenic.

Administration of the methods and compositions described here to an individual results in a reduction in plasma triglyceride levels. Alternatively or in addition, such administration results in an increase in HDL cholesterol levels. Furthermore, or alternatively, reduction of serum apoCIII levels, an increase in PPARγ levels, an increase in PPARγ levels, or all three, result from administering a first agent which is an antagonist of 11β-HSD1 in conjunction with a second agent which comprises a diuretic, preferably an anti-kaliuretic-diuretic, to an individual.

Many patients with metabolic syndrome moreover develop type 2 diabetes (adult-onset diabetes). Type 2 diabetes is characterised by a fasting plasma glucose level of 7.0 mmol/l or higher. Most persons with type 2 diabetes have two metabolic abnormalities that raise the blood glucose to the diabetes range. The first abnormality is insulin resistance; the other is a deficiency in production of insulin by the pancreas.

Type 2 diabetes typically develops when insulin resistance is combined with a mild-to-moderate defect in the secretion of insulin. Insulin resistance thus is a disorder in the metabolism of tissues that interferes with the normal action of insulin to promote glucose uptake and utilisation. It usually precedes the development of type 2 diabetes by many years. There is a close connection between insulin resistance and the risk factors of the metabolic syndrome. The nature of this connection is not fully understood. One factor appears to be an overloading of tissues with fats (lipids). Patients with insulin resistance usually have a high level of free fatty acids, which are released from fat tissue (adipose tissue). When excess fatty acids enter muscle, lipid overload occurs, and this induces insulin resistance. Other factors may contribute to insulin resistance, but tissue overload of lipids appears to be a major factor. This overload in various ways seems to engender the coronary risk factors of the metabolic syndrome.

An elevated blood LDL cholesterol level generally is not considered to be an integral component of the metabolic syndrome. Nevertheless, it is a major independent risk factor that must be present before the other components of the metabolic syndrome can come into play as atherogenic factors. In populations around the world in which the various components of the metabolic syndrome are present, atherosclerotic coronary heart disease is relatively rare when blood LDL levels are very low. In population studies, only when LDL levels begin to rise does the incidence of coronary heart disease begin to increase. Moreover, interventions which lower LDL cholesterol, including administration of HMGCoA reductase inhibitors or fibrates, reduce the prevalence of coronary heart disease. The link between blood LDL levels and insulin resistance has not been extensively studied. Clearly many factors other than insulin resistance contribute to elevated LDL. However, when there is fat overload in the liver, the production of lipoproteins by the liver appears to be increased; this overproduction of lipoproteins containing apolipoprotein B will lead to some rise in LDL levels. For example, obese persons have higher LDL-cholesterol levels than do lean persons. Thus it is not possible to remove elevated LDL entirely from the metabolic syndrome.

Other abnormalities in blood lipids are more characteristic of the metabolic syndrome. There typically are three abnormalities that group together, hence their name, the lipid triad. These include raised triglycerides, small LDL particles, and low HDL cholesterol levels. The lipid triad also has been called the atherogenic lipoprotein phenotype or atherogenic dyslipidemia. Each component of atherogenic dyslipidemia appears to independently promote atherosclerosis. Raised triglycerides indicate the presence of remnant lipoproteins, which seemingly are as atherogenic as LDL. Small LDL slip into the arterial wall more readily than normal-sized triglycerides, and thus have enhanced atherogenicity.

Low HDL probably promotes atherosclerosis in several ways. One notable example is the ability of HDL to remove excess cholesterol from the arterial wall (reverse cholesterol transport); when HDL is low, reverse cholesterol transport is retarded.

A fourth abnormality often accompanies the lipid triad. This is an elevation of apolipoprotein B (apo B). We provide compositions and combinations of a first agent which is an antagonist of 11β-HSD1, together with a second agent which comprises a diuretic, preferably an anti-kaliuretic-diuretic, which decrease plasma concentrations of apolipoprotein B.

Apo B is the major lipoprotein of LDL and triglyceride-rich lipoproteins. Some investigators believe that the total apo B1′ level is the single best indicator for the presence of atherogenic dyslipidemia. Certainly, when total apo B levels are high, a person is at increased risk for coronary heart disease. Patients with insulin resistance often have atherogenic dyslipidemia. When the liver is overloaded with fat, there is an overproduction of apo-B containing lipoproteins. This leads to raised triglycerides, increased remnants lipoproteins, increased total apo B, and small LDL. All of these represent a compensatory response by the liver in its attempt to cope with and remove excess fat.

In addition, an important liver enzyme, hepatic lipase, also is increased in the presence of insulin resistance. This enzyme degrades HDL and contributes to the low HDL associated with insulin resistance.

The glucocorticoid hormones (cortisol, corticosterone) produced by the adrenal gland also have the potential to cause insulin resistance. This action is observed most dramatically in patients who have Cushing's syndromes, such as Cushing's disease, which are due to overproduction of corticosteroids. Patients with Cushing's syndromes manifest insulin resistance, and many develop type 2 diabetes. Moreover, patients who receive natural or synthetic glucocorticoids in treatment of disease also show insulin resistance.

Recently a novel and important level of control of glucocorticoid action has become apparent, pre-receptor metabolism by 11β-hydroxysteroid dehydrogenases (11β-HSDs). 11β-HSDs catalyse the interconversion of active physiological 11-hydroxy glucocorticoids (cortisol in most mammals, corticosterone in rats and mice) and their inert 11-keto forms (cortisone, 11-dehydrocorticosterone). There are two isozymes of 11β-HSD, the products of distinct genes (5, 6). 11β-HSD type 2 is a high affinity dehydrogenase that rapidly inactivates corticosterone in kidney and colon, thus excluding glucocorticoids from otherwise non-selective mineralocorticoid receptors in vivo (7, 8). However, white adipose tissue solely expresses 11β-HSD type 1 (9), as does the liver where the enzyme is particularly abundant (10, 11).

11β-HSD-1 is a predominant reductase in most intact cells, including hepatocytes (12), adipocytes (13), neurons (14), and in the isolated liver ex vivo (15). This reaction direction regenerates active glucocorticoids within cells from free circulating inert 11-ketosteroids. Mice homozygous for targeted disruption of the 11β-HSD-1 gene are viable, fertile and have normal longevity (16). However, 11βHSD-1 null mice cannot regenerate corticosterone from inert 11-dehydrocorticosterone, indicating this isozyme is the unique 11β-reductase. Strikingly, the null animals exhibit attenuated gluconeogenic responses upon stress and resist the hyperglycaemia induced by chronic high fat feeding (16). This occurs despite modestly elevated plasma levels of corticosterone. The results suggest that 11βHSD-1-reductase activity is an important amplifier of intrahepatic glucocorticoid action in vivo. Intriguingly, tissue-specific alterations in 11βHSD-1 activity have been implicated in the development of obesity and insulin resistance in obese Zucker rats (4) and in humans (2; 54).

In the Metabolic Syndrome, dyslipidaemia is characterised by hypertriglyceridaemia and an aberrant lipoprotein and cholesterol profile with elevated VLDL¹, but reduced ‘cardioprotective’ HDL cholesterol (17). The plasma lipid profile is largely determined by gene expression in the liver. Furthermore, expression and activity of many liver proteins involved in lipid metabolism, synthesis, packaging and export are glucocorticoid-sensitive. However, the precise role of glucocorticoids in the pathogenesis of hepatic lipid metabolism is unclear, with overall effects apparently dependent upon steroid concentrations, the levels of other hormones, particularly insulin, and on diet. Indeed, many studies have used short-term treatments and/or non-physiological levels of glucocorticoids, making any extrapolations of the subtle effects of altered intracellular glucocorticoid metabolism difficult. Moreover, glucocorticoids also have important indirect effects, regulating other key transcription factors controlling lipid metabolism, notably inducing the peroxisome proliferator-activated receptor-α (PPARα) (18, 19). PPARα drives the oxidative adaptation to fasting (20, 21) and serves as the molecular target of hypolipidaemic fibrate drugs (22, 23).

The wide range of anti-inflammatory and metabolic effects of the glucocorticoids leads to their use in the treatment of a variety of diseases. The general indications for glucocorticoid therapy include ocular disease, hepatic disorders, malignant haematological disease, solid tumours, intestinal disease, and most prominently immune-mediated and inflammatory-mediated disease. However, glucocorticoid administration is associated with side-effects, which can limit the use of such therapies. Dysregulation of the lipid profile, and the metabolic syndrome, are common side-effects of glucocorticoid administration.

Metabolic syndrome is emerging as one of the major medical and public health problems both in the United States and worldwide. It is characterised by hypertension, hypertriglyceridaemia, and hyperglycaemia, is exacerbated by obesity, and constitutes a risk factor for coronary heart disease.

Coronary heart disease is a condition that manifests as either heart attack (myocardial infarction), heart failure or chest pain (angina pectoris). It is caused by a narrowing and hardening of the coronary arteries (atherosclerosis). One of the primary features of atherosclerosis is the accumulation of cholesterol within the walls of the coronary arteries. Risk factors for coronary heart disease are the underlying causes of atherosclerosis. There are three major causes of coronary atherosclerosis: elevated LDL cholesterol, cigarette smoking, and the metabolic syndrome. Among these LDL cholesterol is the primary cause of atherosclerosis. When the blood level of LDL is increased, atherosclerosis is initiated and sustained. Cigarette smoking and the metabolic syndrome nevertheless constitute significant risk factors.

The metabolic syndrome is composed of individual risk factors that in aggregate greatly raise the risk for coronary heart disease. The metabolic risk factors that make up this syndrome are high triglycerides, small LDL particles, low HDL cholesterol, high blood pressure, high blood glucose, a tendency for blood clotting (thrombosis), and chronic inflammation. Taken in aggregate, these risk factors accelerate the development of atherosclerosis when they occur in the presence of elevated LDL cholesterol. When LDL-cholesterol levels are very low, the risk factors of the metabolic syndrome may have less effect on atherogenesis; but once LDL levels rise, these other risk factors are believed to become increasingly atherogenic.

Many patients with metabolic syndrome moreover develop type 2 diabetes (adult-onset diabetes). Type 2 diabetes is characterised by a fasting plasma glucose level of 7.0 mmol/l or higher. Most persons with type 2 diabetes have two metabolic abnormalities that raise the blood glucose to the diabetes range. The first abnormality is insulin resistance; the other is a deficiency in production of insulin by the pancreas.

Type 2 diabetes typically develops when insulin resistance is combined with a mild-to-moderate defect in the secretion of insulin. Insulin resistance thus is a disorder in the metabolism of tissues that interferes with the normal action of insulin to promote glucose uptake and utilisation. It usually precedes the development of type 2 diabetes by many years. There is a close connection between insulin resistance and the risk factors of the metabolic syndrome. The nature of this connection is not fully understood. One factor appears to be an overloading of tissues with fats (lipids). Patients with insulin resistance usually have a high level of free fatty acids, which are released from fat tissue (adipose tissue). When excess fatty acids enter muscle, lipid overload occurs, and this induces insulin resistance. Other factors may contribute to insulin resistance, but tissue overload of lipids appears to be a major factor. This overload in various ways seems to engender the coronary risk factors of the metabolic syndrome.

An elevated blood LDL cholesterol level generally is not considered to be an integral component of the metabolic syndrome. Nevertheless, it is a major independent risk factor that must be present before the other components of the metabolic syndrome can come into play as atherogenic factors. In populations around the world in which the various components of the metabolic syndrome are present, atherosclerotic coronary heart disease is relatively rare when blood LDL levels are very low. In population studies, only when LDL levels begin to rise does the incidence of coronary heart disease begin to increase. Moreover, interventions which lower LDL cholesterol, including administration of HMGCoA reductase inhibitors or fibrates, reduce the prevalence of coronary heart disease. The link between blood LDL levels and insulin resistance has not been extensively studied. Clearly many factors other than insulin resistance contribute to elevated LDL. However, when there is fat overload in the liver, the production of lipoproteins by the liver appears to be increased; this overproduction of lipoproteins containing apolipoprotein B will lead to some rise in LDL levels. For example, obese persons have higher LDL-cholesterol levels than do lean persons. Thus it is not possible to remove elevated LDL entirely from the metabolic syndrome.

Other abnormalities in blood lipids are more characteristic of the metabolic syndrome. There typically are three abnormalities that group together, hence their name, the lipid triad. These include raised triglycerides, small LDL particles, and low HDL cholesterol levels. The lipid triad also has been called the atherogenic lipoprotein phenotype or atherogenic dyslipidemia. Each component of atherogenic dyslipidemia appears to independently promote atherosclerosis. Raised triglycerides indicate the presence of remnant lipoproteins, which seemingly are as atherogenic as LDL. Small LDL slip into the arterial wall more readily than normal-sized triglycerides, and thus have enhanced atherogenicity.

Low HDL probably promotes atherosclerosis in several ways. One notable example is the ability of HDL to remove excess cholesterol from the arterial wall (reverse cholesterol transport); when HDL is low, reverse cholesterol transport is retarded.

A fourth abnormality often accompanies the lipid triad. This is an elevation of apolipoprotein B (apo B). Apo B is the major lipoprotein of LDL and triglyceride-rich lipoproteins. Some investigators believe that the total apo B1′ level is the single best indicator for the presence of atherogenic dyslipidemia. Certainly, when total apo B levels are high, a person is at increased risk for coronary heart disease. Patients with insulin resistance often have atherogenic dyslipidemia. When the liver is overloaded with fat, there is an overproduction of apo-B containing lipoproteins. This leads to raised triglycerides, increased remnants lipoproteins, increased total apo B, and small LDL. All of these represent a compensatory response by the liver in its attempt to cope with and remove excess fat.

In addition, an important liver enzyme, hepatic lipase, also is increased in the presence of insulin resistance. This enzyme degrades HDL and contributes to the low HDL associated with insulin resistance.

The glucocorticoid hormones (cortisol, corticosterone) produced by the adrenal gland also have the potential to cause insulin resistance. This action is observed most dramatically in patients who have Cushing's syndromes, such as Cushing's disease, which are due to overproduction of corticosteroids. Patients with Cushing's syndromes manifest insulin resistance, and many develop type 2 diabetes. Moreover, patients who receive natural or synthetic glucocorticoids in treatment of disease also show insulin resistance.

Recently a novel and important level of control of glucocorticoid action has become apparent, pre-receptor metabolism by 11β-hydroxysteroid dehydrogenases (11β-HSDs). 11β-HSDs catalyse the interconversion of active physiological 11-hydroxy glucocorticoids (cortisol in most mammals, corticosterone in rats and mice) and their inert 11-keto forms (cortisone, 11-dehydrocorticosterone). There are two isozymes of 11β-HSD, the products of distinct genes (5, 6). 11β-HSD type 2 is a high affinity dehydrogenase that rapidly inactivates corticosterone in kidney and colon, thus excluding glucocorticoids from otherwise non-selective mineralocorticoid receptors in vivo (7, 8). However, white adipose tissue solely expresses 11β-HSD type 1 (9), as does the liver where the enzyme is particularly abundant (10, 11).

11β-HSD-1 is a predominant reductase in most intact cells, including hepatocytes (12), adipocytes (13), neurons (14), and in the isolated liver ex vivo (15). This reaction direction regenerates active glucocorticoids within cells from free circulating inert 11-ketosteroids. Mice homozygous for targeted disruption of the 11βHSD-1 gene are viable, fertile and have normal longevity (16). However, 11βHSD-1 null mice cannot regenerate corticosterone from inert 11-dehydrocorticosterone, indicating this isozyme is the unique 11β-reductase. Strikingly, the null animals exhibit attenuated gluconeogenic responses upon stress and resist the hyperglycaemia induced by chronic high fat feeding (16). This occurs despite modestly elevated plasma levels of corticosterone. The results suggest that 11βHSD-1-reductase activity is an important amplifier of intrahepatic glucocorticoid action in vivo. Intriguingly, tissue-specific alterations in 11βHSD-1 activity have been implicated in the development of obesity and insulin resistance in obese Zucker rats (4) and in humans (2; 54).

In the Metabolic Syndrome, dyslipidaemia is characterised by hypertriglyceridaemia and an aberrant lipoprotein and cholesterol profile with elevated VLDL¹, but reduced ‘cardioprotective’ HDL cholesterol (17). The plasma lipid profile is largely determined by gene expression in the liver. Furthermore, expression and activity of many liver proteins involved in lipid metabolism, synthesis, packaging and export are glucocorticoid-sensitive. However, the precise role of glucocorticoids in the pathogenesis of hepatic lipid metabolism is unclear, with overall effects apparently dependent upon steroid concentrations, the levels of other hormones, particularly insulin, and on diet. Indeed, many studies have used short-term treatments and/or non-physiological levels of glucocorticoids, making any extrapolations of the subtle effects of altered intracellular glucocorticoid metabolism difficult. Moreover, glucocorticoids also have important indirect effects, regulating other key transcription factors controlling lipid metabolism, notably inducing the peroxisome proliferator-activated receptor-α (PPARα) (18, 19). PPARα drives the oxidative adaptation to fasting (20, 21) and serves as the molecular target of hypolipidaemic fibrate drugs (22, 23).

The wide range of anti-inflammatory and metabolic effects of the glucocorticoids leads to their use in the treatment of a variety of diseases. The general indications for glucocorticoid therapy include ocular disease, hepatic disorders, malignant haematological disease, solid tumours, intestinal disease, and most prominently immune-mediated and inflammatory-mediated disease. However, glucocorticoid administration is associated with side-effects, which can limit the use of such therapies. Dysregulation of the lipid profile, and the metabolic syndrome, are common side-effects of glucocorticoid administration.

Other Uses

The compositions disclosed here, as well as the treatment modalities, may be used for other purposes.

For example, a composition comprising a first agent which is an antagonist of 11β-HSD1 and a second agent which comprises a diuretic preferably an anti-kaliuretic-diuretic may be used to achieve or promote any of the following purposes as set out in Table 2 below. Furthermore, administration of a first agent which is an antagonist of 11β-HSD1 simultaneously or sequentially with a second agent which comprises a diuretic, preferably an anti-kaliuretic-diuretic, to an individual may be used to achieve any of these purposes.

TABLE 2
Uses of composition described

increasing insulin sensitivity of an individual
promoting glucose tolerance of an individual
promotion of an atheroprotective lipid profile in an individual
improvement of a lipid profile
reducing cardiovascular disease risk in an individual
increasing insulin sensitivity risk of an individual
increasing metabolic rate of an individual
preventing or reversing an undesired increase in body weight
preventing or reversing an undesired increase in body weight, in which
the composition is administered simultaneously or sequentially with an
appetite suppressant, or an antiobesity drug, or both
reducing an intrahepatic fat level
treatment of inflammation of an individual
promotion of an atheroprotective lipid profile in an individual
promotion of an atheroprotective lipid profile in an individual by a
reduction in plasma triglyceride levels, an increase in HDL cholesterol
levels, reduction of serum apoCIII levels, an increase in PPARα levels,
an increase in PPARγ levels.
prevention of a side-effect of glucocorticoid therapy
prevention of a side-effect of glucocorticoid therapy associated with
cardiovascular risk, altered lipid profile, insulin resistance,
hyperglycaemia, obesity and/or hypertension.
reducing cholesterol storage in a macrophage
reduction of intrahepatic fat levels in an individual
reducing hepatic glucose production
reducing fasting plasma glucose concentrations

Further Aspects

Further aspects of the methods and compositions described here are set out in the following paragraphs in this section, some of which are numbered. It is to be understood that the invention includes each of these aspects as set out below.

Paragraph 1. A composition comprising a first agent which is an antagonist of 11β-HSD1, together with a second agent which comprises an anti-kaliuretic-diuretic.

Paragraph 2. A composition according to Paragraph 1, in which the first agent comprises an inhibitor of 11β-HSD1 transcription, translation, expression, synthesis or activity, or in which the first agent is capable of lowering levels of 11-HSD1.

Paragraph 3. A composition according to Paragraph 1 or Paragraph 2, in which the first agent is selected from the group consisting of: carbenoxolone, 11-oxoprogesterone, 3α,17,21-trihydoxy-5β-pregnan-3-one, 21-hydroxy-pregn-4-ene-3,11,20-trione, androst-4-ene-3,11,20-trione and 3β-hydroxyandrost-5-en-17-one.

Paragraph 4. A composition according to any preceding Paragraph, in which the first agent is carbenoxolone.

Paragraph 5. A composition according to any preceding Paragraph, in which the second agent is capable of modulating an interaction between the first agent and 11β-HSD2, preferably capable of down-regulating an antagonistic effect of the first agent on 11β-HSD2.

Paragraph 6. A composition according to any preceding Paragraph, in which the second agent is not capable of binding to mineralocorticoid receptors.

Paragraph 7. A composition according to Paragraph 5, in which the second agent is capable of preventing renal mineralocorticoid excess.

Paragraph 8. A composition according to any preceding Paragraph, in which the second agent comprises a pyrazine-carbonyl-guanidine.

Paragraph 9. A composition according to any preceding Paragraph, in which the second agent comprises amiloride (3,5-diamino-6-chloro-N-(diaminomethylene) pyrazinecarboxamide), or a salt or ester thereof, preferably amiloride-HCl, more preferably amiloride-monohydrochloride, dihydrate.

Paragraph 10. A composition according to any of Paragraphs 1 to 5, in which the second agent comprises an aldosterone antagonist.

Paragraph 11. A composition according to any of Paragraphs 1 to 5 or 10, in which the second agent comprises an androstadiene-spiro-furan.

Paragraph 12. A composition according to any of Paragraphs 1 to 5, 10 or 11, in which the second agent comprises spironolactone (17-hydroxy-7alpha-mercapto-3-oxo-17alpha-pregn-4-ene-21-carboxylic acid gamma-lactone) or a salt or ester thereof, preferably spironolactone-acetate, or Eplerenone.

Paragraph 13. A pharmaceutical composition comprising a composition according to any preceding Paragraph, together with a pharmaceutically acceptable carrier, excipient or diluent.

Paragraph 14. A composition according to any preceding Paragraph, which is provided in a slow-release formulation.

Paragraph 15. Use of a composition according to any of Paragraphs 1 to 14, for any one or more of the purposes as set out in Table 2.

Paragraph 16. A method of improving any one or more of verbal fluency, verbal memory and logical memory in an individual, which method comprises administering to an individual a first agent as set out in any of Paragraphs 1 to 4, simultaneously or sequentially with a second agent as set out in any of Paragraphs 5 to 12.

Paragraph 17. A method according to Paragraphs 16, which method comprises administering to an individual a therapeutically effective amount of a composition according to any of Paragraphs 1 to 14.

Paragraph 18. A method of treatment of mild cognitive impairment (MCI) in an individual, which method comprises administering to an individual a first agent as set out in any of Paragraphs 1 to 4, simultaneously or sequentially with a second agent as set out in any of Paragraphs 5 to 12.

Paragraph 19. A method of treatment according to Paragraph 18, which method comprises administering to an individual a therapeutically effective amount of a composition according to any of Paragraphs 1 to 14.

Paragraph 20. A composition according to any of Paragraphs 1 to 14, for use in a method of improving verbal fluency or verbal memory or logical memory, or for treatment of mild cognitive impairment (MCI) in an individual.

Paragraph 21. Use of a first agent as set out in any of Paragraphs 1 to 4 in combination with a second agent as set out in any of Paragraphs 5 to 12, or use of a composition according to any of Paragraphs 1 to 14, for the preparation of a composition for the treatment of mild cognitive impairment, or for the preparation of a composition for improvement of verbal fluency, verbal memory, or logical memory.

Paragraph 22. A method or use or a first or medical use according to any of Paragraphs 15 to 21, in which the first agent is administered at a rate of about 4.5 mg/kg/day.

Paragraph 23. A method or use or a first or medical use according to any of Paragraphs 15 to 22, in which the second agent is administered at a rate of about 0.15 mg/kg/day.

Paragraph 24. A method or use or a first or second medical use according to any of Paragraphs 15 to 23, in which verbal fluency is significantly improved as assessed by a Controlled Word Association test, or in which verbal memory is significantly improved as assessed by a Rey Auditory-Verbal Learning Test, or in which logical memory is significantly improved as assessed by a Wechsler Memory Scale.

Paragraph 25. A kit comprising a first agent which is an antagonist of 11β-HSD1, and a second agent which comprises an anti-kaliuretic-diuretic, together with instructions for administration of the agents to an individual with mild cognitive impairment.

We further disclose a composition comprising a first agent which is an antagonist of 11β-HSD1, together with a second agent which comprises a diuretic, preferably an antikaliuretic-diuretic. In an alternative embodiment, we disclose a composition comprising a first agent which is an antagonist of 11β-HSD1, together with a second agent which is capable of modulating (preferably antagonising) an interaction between the first agent and 11β-HSD2.

Preferably, the first agent comprises an inhibitor of 11β-HSD1 transcription, translation, expression, synthesis or activity, or in which the first agent is capable of lowering levels of 11β-HSD1. More preferably, the first agent is selected from the group consisting of: carbenoxolone, 11-oxoprogesterone, 3α,17,21-trihydoxy-5β-pregnan-3-one, 21-hydroxy-pregn-4-ene-3,11,20-trione, androst-4-ene-3,11,20-trione and 3β-hydroxyandrost-5-en-17-one. In a preferred embodiment, the first agent is carbenoxolone.

Preferably, the second agent is capable of modulating an interaction between the first agent and 11β-HSD2, preferably capable of down-regulating an antagonistic effect of the first agent on 11β-HSD2. More preferably, the second agent is capable of preventing renal mineralocorticoid excess. In a preferred embodiment, the second agent comprises an antikaliuretic-diuretic agent. Preferably, the second agent comprises a pyrazine-carbonyl-guanidine.

In a preferred embodiment, the second agent comprises amiloride (3,5-diamino-6-chloro-N-(diaminomethylene) pyrazinecarboxamide. The second agent may comprise a salt or ester of amiloride, preferably amiloride-HCl, more preferably amiloride-monohydrochloride, dihydrate.

The second agent may alternatively or in addition comprise an aldosterone antagonist. Preferably, the second agent comprises an androstadiene-spiro-furan. In a preferred embodiment, the second agent comprises spironolactone (17-hydroxy-7alpha-mercapto-3-oxo-17alpha-pregn-4-ene-21-carboxylic acid gamma-lactone) or a salt or ester thereof, preferably spironolactone-acetate.

We disclose a pharmaceutical composition comprising such a composition, together with a pharmaceutically acceptable carrier, excipient or diluent. Preferably, the composition is provided in a slow-release formulation.

We disclose use of such a composition, for improving verbal fluency, verbal memory or logical memory in an individual, and/or for any one or more of the purposes as set out in Table 2.

We further disclose a method of improving verbal fluency, verbal memory or logical memory in an individual, which method comprises administering to an individual a first agent as set out above, simultaneously or sequentially with a second agent as set out above. Preferably, the method comprises administering to an individual a therapeutically effective amount of a composition as described.

We further disclose a method of treatment of mild cognitive impairment (MCI) in an individual, which method comprises administering to an individual a first agent as set out above, simultaneously or sequentially with a second agent as set out above.

Preferably, the method comprises administering to an individual a therapeutically effective amount of a composition as described.

We disclose a composition as described for use in a method of improving verbal fluency, verbal memory or logical memory, or for treatment of mild cognitive impairment (MCI) in an individual.

We further disclose use of a first agent as set out abovein combination with a second agent as set out above, or use of a composition, for the preparation of a composition for the treatment of mild cognitive impairment, or for the preparation of a composition for improvement of verbal fluency, verbal memory, or logical memory.

Preferably, the first agent is administered at 100 mg every 8 hours, preferably over a course of 4 weeks or more. Preferably, the second agent is administered at 10 mg per day, preferably over a course of 4 weeks or more.

Most preferably, the method, etc is such that verbal fluency is significantly improved as assessed by a Controlled Word Association test, or in which verbal memory is significantly improved as assessed by a Rey Auditory-Verbal Learning Test, or in which logical memory is significantly improved as assessed by a Wechsler Memory Scale.

We further disclose a kit comprising a first agent which is an antagonist of 11β-HSD1, and a second agent which comprises a diuretic, preferably an anti-kaliuretic-diuretic, together with instructions for administration of the agents to an individual with mild cognitive impairment.

We also disclose a method of treatment of a human or animal patient suffering from a condition selected from the group consisting of: hepatic insulin resistance, adipose tissue insulin resistance, muscle insulin resistance, neuronal loss or dysfunction due to glucocorticoid potentiated neurotoxicity, obesity and any combination of the aforementioned conditions, the method comprising the step of administering to said patient a medicament comprising a pharmaceutically active amount of a first agent which is an antagonist of 11β-HSD1, simultaneously or sequentially with a second agent which comprises a diuretic, preferably an antikaliuretic-diuretic.

### EXAMPLES

### Example 1

### Expression of 11β-HSD1

Post-mortem sections (n=3-4/region) of human hippocampus, cerebellum and frontal cortex are obtained with ethical approval and relatives' consent from the Edinburgh Brain Bank. The subjects have no evidence of CNS disorders.

The subjects are 2 women and 2 men (mean 77 y, range 67 to 86) who died of lung carcinoma, oesophageal adenocarcinoma or cardiac failure (2) and had no ante- or postmortem evidence of CNS disorders. Brain sections are taken and processed broadly as previously described [Seckl J R, Dickson K L, Yates C and Fink G (1991). Distribution of glucocorticoid and mineralocorticoid receptor messenger RNA expression in human postmortem hippocampus. Brain Research 561: 332-337.].

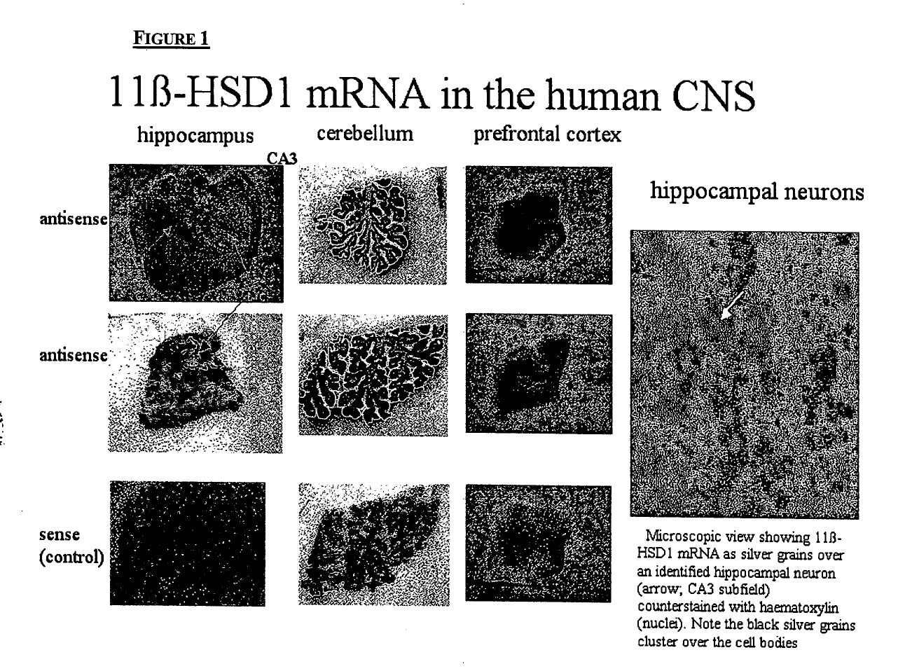

The results are shown in FIG. 1.

11β-HSD1 mRNA is detected in frozen sections using ³⁵S-UTP-labelled antisense cRNA probes transcribed in vitro from a 900 bp HindIII-Sstl fragment of ph11β-HSD1 [Tannin G et al, 1991 266: 16653-8] subcloned in pGEM3. Sections are postfixed in 4% paraformaldehyde, acetylated (0.25% acetic anhydride in 0.1M triethanolamine, pH 8.0), washed in phosphate-buffered saline, dehydrated through graded alcohols and air-dried. Hybridisation with the cRNA probe is carried out as previously described [Yau et al; 1999 Mol Brain Res, 70: 282-287]. Slides are dehydrated, dipped in photographic emulsion (NTB-2, Kodak, UK) and exposed at 4° C. for 6 weeks before developing and counterstaining with 1% pyronine. Control sections are hybridised with identically-labelled sense RNA probes.

11β-HSD1 mRNA is detected by in situ hybridisation using ³⁵S-labelled cRNA in hippocampal neurons (dentate gyrus and comu ammonis), prefrontal cortex and cerebellar granule cell layer. No signal is detected with similarly labelled ‘sense’ control RNA.

Thus, 11β-HSD1 mRNA is expressed in human brain, notably in hippocampus and frontal cortex.

### Example 2

### Cognitive Performance Increase on Administration of Carbenoxolone and Amiloride

10 healthy, unmedicated men (65.5 plus/minus SD 5.5y) participated in a randomised, double-blind, crossover trial comparing carbenoxolone (100 mg 8 hourly for 4 weeks by mouth) plus amiloride (10 mg daily by mouth) with placebo (8 hourly for 4 weeks by mouth) plus amiloride (10 mg daily by mouth). Each study phase lasts 4 weeks.

The two phases are separated by an 8 week washout period. The order of carbenoxolone or placebo is randomised and the subjects and the experimenters are blind to the treatment given. Participants are asked to look out for potential adverse effects of carbenoxolone, including weight gain and pedal edema, and blood pressure and plasma electrolytes are monitored weekly. Compliance is assessed by pill counting and detecting carbenoxolone in plasma by high performance liquid chromatography.

At the end of each phase, tests of verbal fluency, verbal memory, visuospatial memory, attention and processing speed, and intelligence are performed.

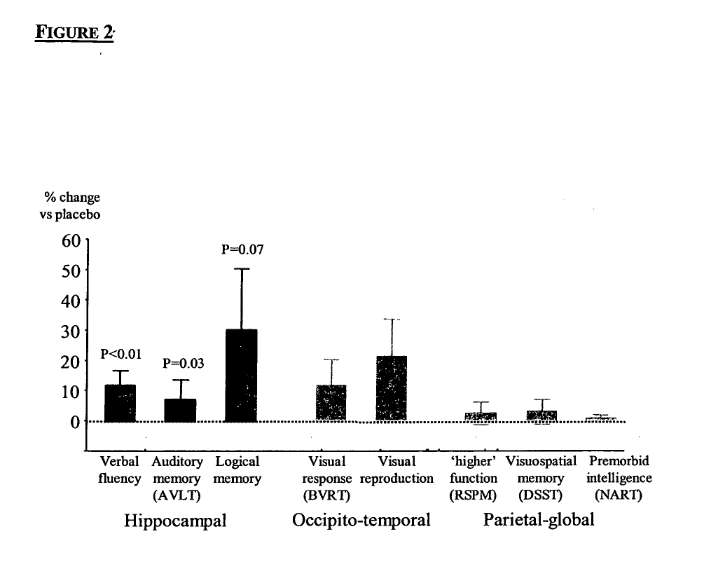

The results are shown in FIG. 2.

Patients who are administered carbenoxolone together with amiloride are found to have improved verbal fluency (Controlled Word Association test; 41/12 vs 44/11, mean/SD, p<0.01). In addition, they are found to have improved verbal memory (Rey Auditory-Verbal Learning Test: p<0.03 by ANOVA). No adverse effects are reported with carbenoxolone plus amiloride. Blood pressure, plasma sodium and potassium levels do not differ between the control and the carbenoxolone (plus amiloride) phases of the study.

Patients are also seen to have improved logical memory.

Data are mean+/−SEM for the % change in cognitive score, calculated for each individual as (score with carbenoxolone+amiloride)−(score with placebo+amiloride)/(score with placebo+amiloride)×100. P values refer to comparison of absolute data in the two groups. Statistical analysis is conducted using Mann-Witney U tests and Friedman's non-parametric ANOVAs.

Administration of carbenoxolone for just 4 weeks to healthy men improved aspects of cognitive function associated with hippocampal and frontal function. This most likely reflects reduced regeneration of cortisol from cortisone by 11β-HSD1 within brain subregions. Inhibition of 11β-HSD1 therefore provides an exciting new therapeutic target to prevent/ameliorate age-associated cognitive dysfunction in humans.

### Example 3

### Cognitive Performance Increase on Administration of Carbenoxolone and Amiloride in Type 2 Diabetes Patients

12 patients with type 2 diabetes by World Health Organisation criteria (age range 52-70 y, mean 60.1 y), including 3 women and 9 men, participated in a randomised double-blind placebo controlled crossover trial comparing carbenoxolone (100 mg 8 hourly for 6 weeks by mouth) plus amiloride (10 mg daily by mouth) with placebo (8 hourly for 6 weeks by mouth) plus amiloride (10 mg daily by mouth).

The two phases are separated by a washout period of 8 weeks. The order of carbenoxolone or placebo is randomised and the subjects and the experimenters are blind to the treatment given.

At the end of each phase, tests of verbal fluency, verbal memory, visuospatial memory, attention and processing speed and intelligence are performed.

The results are shown in Table 3 below.

Patients who were administered carbenoxolone together with amiloride are found to have improved verbal memory (Rey Auditory-Verbal Learning Test), p<0.01).

TABLE 3
Improvement in cognitive function with carbenoxolone plus amiloride
in patients with type 2 diabetes mellitus. P values refer to Mann-
Whitney U tests; WM - Wechsler Memory Scale-revised; AVLT - Rey
Auditory Verbal Learning Test; RSPM - Ravens Standard
Progressive Matrices; DSST - Digit Symbol Substitution test.

| | | Placebo with | Carbenoxolone | |
|---|---|---|---|---|
| | Neuropsychological | Amiloride | with Amiloride | |
| Domain | measures | Mean (SD) | Mean (SD) | p |
| Executive | Verbal Fluency | 42.2 (8.4) | 42.7 (6.4) | 0.48 |
| function | | | | |
| Memory | | | | |
| Visual | WM Visual | 59.9 (13.3) | 60.2 (7.9) | 0.94 |
| | reproduction | | | |
| Verbal | WM Logical | 49.7 (13.8) | 49.7 (17.7) | 0.78 |
| | Memory | | | |
| | Rey AVLT | 56.1 (8.7) | 59.7 (5.6) | <0.01 |
| Reasoning | RSPM | 44.0 (6.6) | 45.4 (8.1) | 0.17 |
| Processing | DSST | 50.3 (6.7) | 50.7 (5.5) | 0.78 |
| speed | | | | |

The invention will now be further described by the following numbered paragraphs:

1. A composition comprising a first agent comprising an antagonist of 11β-HSD1, together with a second agent comprising an anti-kaliuretic-diuretic.

2. A composition according to Paragraph 1, in which the first agent comprises an inhibitor of 11β-HSD1 transcription, translation, expression, synthesis or activity, or in which the first agent is capable of lowering levels of 11β-HSD1.

3. A composition according to Paragraph 1 or Paragraph 2, in which the first agent is selected from the group consisting of: carbenoxolone, 11-oxoprogesterone, 3α,17,21-trihydoxy-5β-pregnan-3-one, 21-hydroxy-pregn-4-ene-3,11,20-trione, androst-4-ene-3,11,20-trione and 3β-hydroxyandrost-5-en-17-one.

4. A composition according to any preceding paragraph, in which the first agent comprises carbenoxolone.

5. A composition according to any preceding paragraph, in which the second agent is capable of modulating an interaction between the first agent and 11β-HSD2, preferably capable of down-regulating an antagonistic effect of the first agent on 11β-HSD2.

6. A composition according to any preceding paragraph, in which the second agent is not capable of binding to a mineralocorticoid receptor.

7. A composition according to Paragraph 5, in which the second agent is capable of preventing renal mineralocorticoid excess.

8. A composition according to any preceding paragraph, in which the second agent comprises a pyrazine-carbonyl-guanidine.

9. A composition according to any preceding paragraph, in which the second agent comprises amiloride (3,5-diamino-6-chloro-N-(diaminomethylene) pyrazinecarboxamide), or a salt or ester thereof, preferably amiloride-HCl, more preferably amiloride-monohydrochloride, dihydrate.

10. A composition according to any of Paragraphs 1 to 5, in which the second agent comprises an aldosterone antagonist.

11. A composition according to any of Paragraphs 1 to 5 or 10, in which the second agent comprises an androstadiene-spiro-furan.

12. A composition according to any of Paragraphs 1 to 5, 10 or 11, in which the second agent comprises spironolactone (17-hydroxy-7alpha-mercapto-3-oxo-17alpha-pregn-4-ene-21-carboxylic acid gamma-lactone) or a salt or ester thereof, preferably spironolactone-acetate, or Eplerenone.

13. A pharmaceutical composition comprising a composition according to any preceding paragraph, together with a pharmaceutically acceptable carrier, excipient or diluent.

14. A composition according to any preceding paragraph, which is provided in a slow-release formulation.

15. A composition according to any of Paragraphs 1 to 14, for use in a method of improving verbal fluency, verbal memory or logical memory, or any combination thereof, in an individual.

16. A composition according to any of Paragraphs 1 to 14, for use in a method of treatment or prevention of mild cognitive impairment (MCI) in an individual.

17. A composition according to Paragraph 15 or 16 for a use as specified therein, in which the individual is suffering from Type 2 diabetes.

18. A first agent comprising an antagonist of 11β-HSD1 for use in a method of improving verbal fluency, verbal memory or logical memory, or any combination thereof, in an individual, in which the method comprises administering an 11β-HSD1 antagonist simultaneously or sequentially with a second agent comprising an anti-kaliuretic-diuretic.

19. A second agent comprising an anti-kaliuretic-diuretic for use in a method of improving verbal fluency, verbal memory or logical memory, or any combination thereof, in an individual, in which the method comprises administering an anti-kaliuretic-diuretic simultaneously or sequentially with a first agent comprising an antagonist of 11β-HSD1.

20. A first agent comprising an antagonist of 11β-HSD1 for use in a method of treatment or prevention of Mild Cognitive Impairment (MCI) in an individual, in which the method comprises administering an 11β-HSD1 antagonist simultaneously or sequentially with a second agent comprising an anti-kaliuretic-diuretic.

21. A second agent comprising an anti-kaliuretic-diuretic for use in a method of treatment or prevention of Mild Cognitive Impairment (MCI) in an individual, in which the method comprises administering an anti-kaliuretic-diuretic simultaneously or sequentially with a first agent comprising an antagonist of 11β-HSD1.

22. Use of a first agent comprising an antagonist of 11β-HSD1, together with a second agent comprising an anti-kaliuretic-diuretic, for the preparation of a composition for improvement of verbal fluency, verbal memory, or logical memory, or any combination thereof.

23. Use of a first agent comprising an antagonist of 11β-HSD1, together with a second agent comprising an anti-kaliuretic-diuretic, for the preparation of a composition for the treatment or prevention of Mild Cognitive Impairment (MCI).

24. A use according to any of Paragraphs 18 to 23, in which the first agent has the features as set out in any of Paragraphs 1 to 4, or in which the second agent has the features as set out in any of Paragraphs 5 to 12.

25. An use according to any of Paragraphs 18 to 24, in which verbal fluency is significantly improved as assessed by a Controlled Word Association test, or in which verbal memory is significantly improved as assessed by a Rey Auditory-Verbal Learning Test, or in which logical memory is significantly improved as assessed by a Wechsler Memory Scale.

26. A kit comprising a first agent comprising an antagonist of 11β-HSD1, and a second agent comprising an anti-kaliuretic-diuretic.

27. A kit according to Paragraph 26, in which the first agent and the second agent are in separate containers.

28. A kit according to Paragraph 26 or 27, in which the first agent has the features as set out in any of Paragraphs 1 to 4, or in which the second agent has the features as set out in Paragraphs 2 to 12.

29. A kit according to Paragraph 26, 27 or 28, further comprising instructions for administration of the agents to an individual to improve verbal fluency, verbal memory, or logical memory, or any combination thereof.

30. A kit according to Paragraph 26, 27 or 28, further comprising instructions for administration of the agents to an individual with mild cognitive impairment.

31. A method of preparing a composition according to any of Paragraphs 1 to 14, the method comprising admixing a first agent comprising an antagonist of 11β-HSD1, with a second agent comprising an anti-kaliuretic-diuretic.

32. A method according to Paragraph 31, in which the first agent has the features as set out in any of Paragraphs 2 to 4, or in which the second agent has the features as set out in any of Paragraphs 5 to 12.

33. Use of a composition according to any of Paragraphs 1 to 14, for improving verbal fluency, verbal memory or logical memory, or any combination thereof, in an individual.

34. Use of a composition according to any of Paragraphs 1 to 14, for treating or preventing Mild Cognitive Impairment in an individual.

35. A method of improving any one or more of verbal fluency, verbal memory or logical memory, or any combination thereof, in an individual, which method comprises administering to an individual a first agent comprising an antagonist of 11β-HSD1, simultaneously or sequentially with a second agent comprising an anti-kaliuretic-diuretic.

36. A method of treatment or prevention of mild cognitive impairment (MCI) in an individual, which method comprises administering to an individual a first agent comprising an antagonist of 11β-HSD1, simultaneously or sequentially with a second agent comprising an anti-kaliuretic-diuretic.

37. A method according to Paragraph 35 or 36, in which the first agent has the features as set out in any of Paragraphs 2 to 4, or in which the second agent has the features as set out in any of Paragraphs 5 to 12.

38. A method or use according to any of Paragraphs 33 to 37, which method comprises administering to an individual a therapeutically effective amount of a composition according to any of Paragraphs 1 to 14.

39. A method or use according to any of Paragraphs 33 to 38, in which the first agent is administered at a rate of about 4.5 mg/kg/day.

40. A method or use according to any of Paragraphs 33 to 39, in which the second agent is administered at a rate of about 0.15 mg/kg/day.

41. A method or use according to any of Paragraphs 33 to 40, in which the individual is suffering from Type 2 diabetes.

42. A method of treatment of a human or animal patient suffering from a condition selected from the group consisting of: hepatic insulin resistance, adipose tissue insulin resistance, muscle insulin resistance, neuronal loss or dysfunction due to glucocorticoid potentiated neurotoxicity, obesity and any combination of the aforementioned conditions, the method comprising the step of administering to said patient a medicament comprising a pharmaceutically active amount of a first agent which is an antagonist of 11 P—HSD1, simultaneously or sequentially with a second agent which comprises a diuretic, preferably an antikaliuretic-diuretic.

43. Use of a composition according to any of Paragraphs 1 to 14, for any one or more of the purposes as set out in Table 2.

### REFERENCES

1. Peeke, P. M. and Chrousos, G. P. (1995) Ann. NY. Acad. Sci. 771, 665-676.
2. Andrew, R., Phillips, D. I. W. and Walker, B. R. (1998) J. Clin. Endocrinol. Metab. 83, 1806-1809.
3. Stewart, P. M., Boulton, A., Kumar, S., Clark, P. M. S. and Shackleton, C. H. L. (1999) J. Clin. Endocrinol. Metab. 84, 1022-1027.
4. Livingstone, D. E., Jones, G. C., Smith, K., Jamieson, P. M., Andrew, R, Kenyon, C. J. and Walker, B. R. (2000) Endocrinol. 141, 560-563.
5. Brown, R. W., Chapman, K. E., Edwards, C. R. W. and Seckl, J. R. (1993) Endocrinol. 132, 2614-2621. Human Placental 11b-hydroxysteroid dehydrogenase:
6. Albiston A. L., Obeyesekere, V. R., Smith, R. E., Krozowski, Z. S. (1994) Mol. Cell. Endo. 105, R11-R17.
7. Edwards, C. R. W., Stewart, P. M., Burt, D., Brett, L., McIntyre, M. A., Sutanto, W. S. de Kloet, E. R. and Monder, C. (1988) Lancet October 29;2(8618):986-989.
8. Funder, J. W., Pearce, P. T., Smith, R. and Smith, A. I. (1988) Science 242, 583-585
9. Bujalska, I. J., Kumar, S. and Stewart, P. M. (1997) Lancet 349 (9060)1210-1213. Does central obesity reflect Cushing's disease of the omentum?
10. Agarwal, A. K., Monder, C., Eckstein, B. and White, P. C. (1989) J. Biol. Chem. 264, 18939-18943.
11. Rajan, V., Chapman, K. E., Lyons, V., Jamieson, P. M., Mullins, J. J., Edwards, C. R. W. and Seckl, J. R. (1995) J. Steroid. Biochem. Molec. Biol. 52, 141-147.
12. Jamieson, P. M., Chapman, K. E., Edwards, C. R. W. and Seckl, J. R. (1995) Endocrinol. 136, 4754-4761.
13. Napolitano, A., Voice, M., Edwards, C. R. W., Seckl, J. R. and Chapman, K. E. (1998) J. Steroid Biochem. Molec. Biol. 64, 251-260.
14. Rajan, V., Edwards, C. R. W. and Seckl, J. R. (1996) J. Neurosci. 16, 65-70.
15. Jamieson, P. M., Walker, B. R. Chapman, K. E., Andrew, R., Rossiter, S. and Seckl, J. R. (2000) J. Endocrinol. 165, 685-692. 11
16. Kotelevtsev, Y., Holmes, M. C., Burchell, A., Houston, P. M., Schmoll, D., Jamieson, P., Best, R., Brown, R., Edwards, C. R. W., Seckl, J. R. and Mullins, J. J. (1997) Proc. Natl. Acad. Sci. USA 94, 14924-14929.
17. Grundy, S. M. (1998) Am. J. Cardiol. 81, 18B-25B.
18. Lemberger, T, Staels, B, Saladin, R., Desvergne, B., Auwerx, J. and Wahli, W. (1994) J. Biol. Chem. 269, 24527-24530.
19. Lemberger, T., Saladin, R., Vasquez, M., Assimacopoulos, F., Staels, B., Desvergne, B., Wahli, W and Auwerx, J. (1996) J. Biol. Chem. 271, 1764-1769.
20. Kersten, S., Seydoux, J., Peters, J. M., Gonzalez, F. J., Desvergne, B. and Wahli, W. (1999) J. Clin. Invest. 103, 1489-1498.
21. Leone, T. C., Weinheimer, C. J. and Kelly, D. P (1999) Proc. Natl. Acad. Sci. USA 96, 7473-7478.
22. Krey, G., Braissant, O., L'Horset, F., Kalkhoven, E., Pyrroud, M., Parker, M. G. and Wahli, W. (1997) Mol. Endocrinol. 11, 779-791.
23. Peters, J. M., Hennuyer, N., Staels, B., Fruchart, J.-C., Fievet, C., Gonzalez, F. J. and Auwerx, J. (1997) J. Biol. Chem. 272, 27307-27312.
24. Harris, H. J., Kotelevtsev, Y., Mullins, J. J., Seckl, J. R. and Holmes, M. C. (2001) Endocrinology 2001 142: 114-120
25. Foretz, M., Guichard, C., Ferre, P. and Foufelle, F. (1999) Proc. Natl. Acad. Sci USA 96, 12737-12742.
26. Shimano, H., Yahagi, N., Amemiya-Kudo, M., Hasty, A. H., Osuga, J.-I., Tamura, Y., Shionoiri, F., Iizuka, Y., Ohashi, K., Harada, K., Gotoda, T., Ishibashi, S and Yamada, N. (1999) J. Biol. Chem. 274, 35832-35839.
27. M^{c}Garry, J. D. and Brown, N. F. (1997) Eur. J. Biochem. 244, 1-14.
28. Fan, C. Y., Pan, J., Usuda, N., Yeldandi, A. V., Rao, M. S. and Reddy, J. K. (1998) J. Biol. Chem. 273, 15639-15645.
29. Boss, O., Hagan, T. and Lowell, B. B. (2000) Diabetes 49, 143-156.
30. Kelly, L. J., Vicario, P. P., Thompson, G. M., Candelore, M. R., Doebber, T. W., Ventre, J., Wu, M. S., Meurer, R., Forest, M. J., Conner, M. W., Cascieri, M. A. and Moller, D. E. (1998) Endocrinol. 139, 4920-4927.
31. Yu, G. S., Lu, Y. C. and Gulick, T. (1998) J. Biol. Chem. 273, 32901-32909.
32. Lee, S.-S., Pineau, T., Drago, J., Lee, E. J., Owens, J. W., Kroetz, D. L., Femandez-Salguero, P. M., Westphal, H. and Gonzalez, F. J. (1995) Mol. Cell. Biol. 15, 3012.
33. Tsuboyama-Kasaoka, N., Takahashi, M., Kim, H. and Ezaki, O. (1999) Biochem Biophys. Res. Commun. 257, 879-885.
34. Ebara, T., Ramakrishnan, R., Steiner, G. and Shachter, N. S., (1997) J. Clin. Invest. 99, 2672-2681.
35. Barter, P. J. and Rye, K. A. (1996) Atherosclerosis 121, 1-12.
36. Bhattacharya, A and Holland, L. J. (1991) Mol. Endocrinol. 5(4), 587-597.
37. Kannel, W. B. (1997) Drugs 54, Suppl 3, 32-40.
38. Aalto-Setala, K., Fisher, E. A., Chen, X., Chajek-Shaul, T., Hayek, T., Zechner, R., Walsh, A., Ramakrishnan, R., Ginsberg, H. N. and Breslow, J. L. (1992) J. Clin. Invest. 90, 1889-1890.
39. Ito, Y., Azrolan, N., O'Connell, A., Walsh, A. and Breslow, J. L. (1990) Science 249, 790-793.
40. Dammerman, M., Sankuijl, L. A., Halaas, J. L., Chung, W. and Breslow, J. L. (1993) Proc. Natl. Acad. Sci USA 90, 4562-4566.
41. Kockx, M., Gervois, P. P., Poulain, P., Derudas, B., Peters, J. M., Gonzalez, F. J., Princen, H. M., Kooistra, T and Staels, B. (1999) Blood 93, 2991-2998.
42. Yau et al., (2001) Proc. Natl. Acad. Sci. USA, 98, 4716-4721.
43. Cole, T. J., Harris, H. J., Hoong, I., Solomon, N., Smith, R., Krozowski, Z. and Fullerton, M. J. (1999)
44. Dong, Y., Poellinger, L., Gustafsson, J. A. and Okret, S. (1988) Mol. Endocrinol. 2(12), 1256-1264. Collett, G. P., Betts, A M., Johnson, M. I., Pulimood, A. B., Cook, S., Neal, D. E. and Robson C. N. (2000) Clin. Cancer Res. 6, 3241-3248.
45. Qi, C., Zhu, Y. J. and Reddy, J. K.(2000)Cell Biochem. Biophys 32, 187-204. Valmaseda, A, Carmona, M. C., Barbera, M. J., Vinas, O., Mampel, T., Iglesias, R., Villarroya, F., Giralt, M. 1(999) Mol. Cell. Endocrinol. 154, 101-109.
46. Schultz R., Yan, W., Toppari, J., Volkl, A., Gustafsson, J. A. and Pelto-Huikko, M. (1999) Endocrinol. 140, 2968-2975.
47. Ambrosi, B., Sartorio, A., Pizzocaro, A., Passini, E., Bottasso, B. and Federici, A. (2000) Exp. Clin. Endocrinol. Diab. 108, 294-298.
48. Staels, B., van Tol, A., Chan, L., Verhoeven, G. and Auwerx, J., (1991) Arterioscler Thromb. May-June;11(3):760-9.
49. Masuzaki et al., (2001) Science 294:2166-2170
50. Kim J K, Gavrilova O, Chen Y, Reitman M L, Shulman G I. J Biol Chem 2000; 275: 8456-8460.
51. Gavrilova O, Marcus-Samuels B, Graham D, et al. J Clin Invest 2000; 105: 271-278.
52. Ryysy L, Hakkinen A M, Goto T, et al. Diabetes 2000; 49: 749-758.
53. Marchesini G, Brizi M, Bianchi G, et al. Diabetes 2001; 50: 1844-1850.
54. Rask E, Olsson T, Soderberg S, Andrew R, Livingstone DEW, Johnson O & Walker B R 2001 Journal of Clinical Endocrinology and Metabolism 86 1418-1421.
55. Morton N M, Holmes M C, Fievet C, Staels B, Tailleux A, Mullins J J & Seckl J R 2001 Journal of Biological Chemistry 276 41293-41300.
56. Stewart P M, Wallace A M, Atherden S M, Shearing C H, Edwards C R. Clin Sci (Colch) 1990; 78: 49-54.
57. Best R, Walker B R. Clin Endocrinol (Oxf) 1997; 47: 231-236.
58. DeFronzo R A, Tobin J D, Andres R. Am J Physiol 1979; 237: E214-E223
59. Puhakainen I, Koivisto V A, Yki-Järvinen H. Diabetes 1991; 40: 1319-1327.
60. Steele R. Ann NY Acad Sci 1959; 82: 420-430.
61. Thomsen C, Becker U, Winkler K, Christoffersen P, Jensen M, Henriksen O. Magn Reson Imaging 1994; 12: 487-495.
62. Stenman U-H, Pesonen K, Ylinen K, Huhtala ML, Teramo K. J Chromatogr 1984; 297: 327-332.
63. Kadish A H, Little R L, Sternberg J C. Clin Chem 1968; 14: 116-131.
64. Yki-Järvinen H, Kauppila M, Kujansuu E, et al. N Engl J Med 1992; 327: 1426-1433.
65. Desbuquois B, Aurbach G D. J Clin Endocrinol Metab 1971; 33: 732-738.
66. Miles J, Classcock R, Aikens J, Gerich J, Haymond N. J Lipid Res 1983; 24: 96-99.
67. Lukaski H C, Johnson P E, Bolonchuk W W, Lykken G I. Am J Clin Nutr 1985; 41: 810-817.
68. Stewart P M, Wallace A M, Atherden S M, Shearing C H & Edwards C R W 1990 Clinical Science 78 49-54.
69. Jellinck P H, Monder C, McEwen B S & Sakai R R 1993 Journal of Steroid Biochemistry and Molecular Biology 46 209-213.
70. Latif S A, Conca T J & Morris D J 1990 Steroids 55 52-58.
71. Bestetti G E, Abramo F, Guillaume-Gentil C, Rohner-Jeanrenaud F, Jeanrenaud B & Rossi G L 1990 Endocrinology 126 1880-1887.
72. Tannin, et al. (1991) J. Biol. Chem. 266: 16653-16658.
73. Studier et al, (1990) Methods in Enzymol. 185; 60-89.
74. Al-Dujaili et al. (1981) Steroids 37 157-176.
75. Chawla et al. (2001) Nature Medicine, 7:48-52.
76. Chinetti et al. (2001) Nature Medicine, 7:53-58.
77. Best et al. (1997) Journal of Endocrinology 153, 41-48.
78. Conti et al. (1994) Nephrology, Dialysis, Transplantation 9, 1622-1628.
79. Marks et al. (1982) Journal of Clinical Endocrinology and Metabolism 54, 1075-1077.
80. Noon et al. (1996) British Journal of Dermatology 134, 837-842.
81. Teelucksingh et al. (1990) Lancet 335, 1060-1063.
82. Walker et al. (1992). Endocrinology 131, 970-972.
83. Le May et al. (2000) FEBS Letters 475, 163-166.
84. Forman et al. (1997) PNAS 94, 4312-4317.
85. Murao et al. (1998) Journal of Biological Chemistry 273, 18959-18965.
86. Fan et al. (1994) PNAS 91, 8724-8728.
87. Wang et al. (1998) J. Biol. Chem. 273, 32920-32926.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. A composition comprising a first agent comprising an antagonist of 11β-HSD1, together with a second agent comprising an anti-kaliuretic-diuretic.

2. The composition according to claim 1, in which the first agent comprises an inhibitor of 11β-HSD1 transcription, translation, expression, synthesis or activity, or in which the first agent is capable of lowering levels of 11β-HSD1.

3. The composition according to claim 1, in which the first agent is selected from the group consisting of: carbenoxolone, 11-oxoprogesterone, 3α,17,21-trihydoxy-5β-pregnan-3-one, 21-hydroxy-pregn-4-ene-3,11,20-trione, androst-4-ene-3,11,20-trione and 3-hydroxyandrost-5-en-17-one.

4. The composition according to claim 4, in which the first agent comprises carbenoxolone.

5. The composition according to claim 1, in which the second agent is capable of modulating an interaction between the first agent and 11β-HSD2.

6. The composition according to claim 5, in which the second agent is capable of down-regulating an antagonistic effect of the first agent on 11β-HSD2.

7. The composition according to claim 5, in which the second agent is not capable of binding to a mineralocorticoid receptor.

8. The composition according to claim 5, in which the second agent is capable of preventing renal mineralocorticoid excess.

9. The composition according to claim 1, in which the second agent comprises a pyrazine-carbonyl-guanidine.

10. The composition according to claim 1, in which the second agent comprises amiloride (3,5-diamino-6-chloro-N-(diaminomethylene) pyrazinecarboxamide), or a salt or ester thereof.

11. The composition according to claim 10, wherein the second agent comprises amiloride-HCl.

12. The composition according to claim 10, wherein the second agent comprises amiloride-monohydrochloride, dihydrate.

13. The composition according to claim 1, in which the second agent comprises an aldosterone antagonist.

14. The composition according to claim 1, in which the second agent comprises an androstadiene-spiro-furan.

15. The composition according to claim 1, in which the second agent comprises spironolactone (17-hydroxy-7alpha-mercapto-3-oxo-17alpha-pregn-4-ene-21-carboxylic acid gamma-lactone) or a salt or ester thereof, including spironolactone-acetate, or Eplerenone.

16. The pharmaceutical composition comprising a composition according to claim 1, together with a pharmaceutically acceptable carrier, excipient or diluent.

17. The composition according to claim 1, which is provided in a slow-release formulation.

18. The composition according to claim 1, for use in a method of improving verbal fluency, verbal memory or logical memory, or any combination thereof or of treatment or prevention of mild cognitive impairment (MCI), in an individual.

19. The composition according to claim 18 for a use as specified therein, in which the individual is suffering from Type 2 diabetes.

20. A first agent comprising an antagonist of 11β-HSD1 for use in a method of improving verbal fluency, verbal memory or logical memory, or any combination thereof or in a method of treatment or prevention of Mild Cognitive Impairment (MCI), in an individual, in which the method comprises administering an 11β-HSD1 antagonist simultaneously or sequentially with a second agent comprising an anti-kaliuretic-diuretic.

21. A second agent comprising an anti-kaliuretic-diuretic for use in a method of improving verbal fluency, verbal memory or logical memory, or any combination thereof or in a method of treatment or prevention of Mild Cognitive Impairment (MCI), in an individual, in which the method comprises administering an anti-kaliuretic-diuretic simultaneously or sequentially with a first agent comprising an antagonist of 11β-HSD1.

22. A method of using the first agent of claim 20, together with a second agent comprising an anti-kaliuretic-diuretic, for the preparation of a composition for improvement of verbal fluency, verbal memory, or logical memory, or any combination thereof or for the treatment or prevention of Mild Cognitive Impairment (MCI).

23. The method of claim 22 wherein the first agent comprises an inhibitor of 1β-HSD1 transcription, translation, expression, synthesis or activity, or in which the first agent is capable of lowering levels of 11β-HSD1.

24. The method of claim 22 in which the first agent is selected from the group consisting of: carbenoxolone, 11-oxoprogesterone, 3α,17,21-trihydoxy-5β-pregnan-3-one, 21-hydroxy-pregn-4-ene-3,11,20-trione, androst-4-ene-3,11,20-trione and 3β-hydroxyandrost-5-en-17-one.

25. The method of claim 24, in which the first agent comprises carbenoxolone.

26. The method of claim 22, wherein the second agent is capable of modulating an interaction between the first agent and 11β-HSD2.

27. The method of claim 26, wherein the second agent is capable of down-regulating an antagonistic effect of the first agent on 11β-HSD2.

28. The method of claim 26, wherein the second agent is not capable of binding to a mineralocorticoid receptor.

29. The method of claim 26, wherein the second agent is capable of preventing renal mineralocorticoid excess.

30. The method of claim 22, wherein the second agent comprises a pyrazine-carbonyl-guanidine.

31. The method of claim 22, wherein the second agent comprises amiloride (3,5-diamino-6-chloro-N-(diaminomethylene) pyrazinecarboxamide), or a salt or ester thereof.

32. The method of claim 31, wherein the second agent comprises amiloride-HCl.

33. The method of claim 31, wherein the second agent comprises amiloride-monohydrochloride, dihydrate.

34. The method of claim 22, wherein the second agent comprises an aldosterone antagonist.

35. The method of claim 22, wherein the second agent comprises an androstadiene-spiro-furan.

36. The method of claim 22, wherein the second agent comprises spironolactone (17-hydroxy-7alpha-mercapto-3-oxo-17alpha-pregn-4-ene-21-carboxylic acid gamma-lactone) or a salt or ester thereof.

37. The method of claim 36, wherein the second agent comprises spironolactone-acetate or Eplerenone.

38. The method of use according to claim 22, in which verbal fluency is significantly improved as assessed by a Controlled Word Association test, or in which verbal memory is significantly improved as assessed by a Rey Auditory-Verbal Learning Test, or in which logical memory is significantly improved as assessed by a Wechsler Memory Scale.

39. A kit comprising a first agent comprising an antagonist of 11β-HSD1, and a second agent comprising an anti-kaliuretic-diuretic.

40. A kit according to claim 39, in which the first agent and the second agent are in separate containers.

41. The kit of claim 39 wherein the first agent comprises an inhibitor of 11β-HSD1 transcription, translation, expression, synthesis or activity, or in which the first agent is capable of lowering levels of 11β-HSD1.

42. The kit of claim 39 in which the first agent is selected from the group consisting of: carbenoxolone, 11-oxoprogesterone, 3α,17,21-trihydoxy-5 β-pregnan-3-one, 21-hydroxy-pregn-4-ene-3,11,20-trione, androst-4-ene-3,11,20-trione and 3β-hydroxyandrost-5-en-17-one.

43. The kit of claim 42, in which the first agent comprises carbenoxolone.

44. The kit of claim 39, wherein the second agent is capable of modulating an interaction between the first agent and 11β-HSD2.

45. The kit of claim 44, wherein the second agent is capable of down-regulating an antagonistic effect of the first agent on 11β-HSD2.

46. The kit of claim 44, wherein the second agent is not capable of binding to a mineralocorticoid receptor.

47. The kit of claim 44, wherein the second agent is capable of preventing renal mineralocorticoid excess.

48. The kit of claim 39, wherein the second agent comprises a pyrazine-carbonyl-guanidine.

49. The kit of claim 39, wherein the second agent comprises amiloride (3,5-diamino-6-chloro-N-(diaminomethylene) pyrazinecarboxamide), or a salt or ester thereof.

50. The kit of claim 49, wherein the second agent comprises amiloride-HCl.

51. The kit of claim 49, wherein the second agent comprises amiloride-monohydrochloride, dihydrate.

52. The kit of claim 39, wherein the second agent comprises an aldosterone antagonist.

53. The kit of claim 39, wherein the second agent comprises an androstadiene-spiro-furan.

54. The kit of claim 39, wherein the second agent comprises spironolactone (17-hydroxy-7alpha-mercapto-3-oxo-17alpha-pregn-4-ene-21-carboxylic acid gamma-lactone) or a salt or ester thereof.

55. The method of claim 54, wherein the second agent comprises spironolactone-acetate or Eplerenone.

56. The kit according to claim 39, further comprising instructions for administration of the agents to an individual to improve verbal fluency, verbal memory, or logical memory, or any combination thereof.

57. The kit according to claim 39, further comprising instructions for administration of the agents to an individual with mild cognitive impairment.

58. A method of preparing a composition according to claim 1, the method comprising admixing a first agent comprising an antagonist of 11β-HSD1, with a second agent comprising an anti-kaliuretic-diuretic.

59. A method of using a composition according to claim 1, for improving verbal fluency, verbal memory or logical memory, or any combination thereof or for treating or preventing Mild Cognitive Impairment, in an individual.

60. A method of improving any one or more of verbal fluency, verbal memory or logical memory, or any combination thereof, in an individual, which method comprises administering to an individual a first agent comprising an antagonist of 11β-HSD1, simultaneously or sequentially with a second agent comprising an anti-kaliuretic-diuretic.

61. A method of treatment or prevention of mild cognitive impairment (MCI) in an individual, which method comprises administering to an individual a first agent comprising an antagonist of 11β-HSD1, simultaneously or sequentially with a second agent comprising an anti-kaliuretic-diuretic.

62. The method of claim 60, wherein the first agent and second agent are administered in the form of a therapeutically effective amount of a composition comprising a first agent comprising an antagonist of 11β-HSD1, together with a second agent comprising an anti-kaliuretic-diuretic.

63. The method of claim 61, wherein the first agent and second agent are administered in the form of a therapeutically effective amount of a composition comprising a first agent comprising an antagonist of 11β-HSD1, together with a second agent comprising an anti-kaliuretic-diuretic.

64. The method of claim 59, in which the first agent is administered at a rate of about 4.5 mg/kg/day.

65. The method of claim 60, in which the first agent is administered at a rate of about 4.5 mg/kg/day.

66. The method of claim 61, in which the first agent is administered at a rate of about 4.5 mg/kg/day.

67. The method of claim 59, in which the second agent is administered at a rate of about 0.15 mg/kg/day.

68. The method of claim 60, in which the second agent is administered at a rate of about 0.15 mg/kg/day.

69. The method of claim 61, in which the second agent is administered at a rate of about 0.15 mg/kg/day.

70. The method of claim 59, in which the individual is suffering from Type 2 diabetes.

71. The method of claim 60, in which the individual is suffering from Type 2 diabetes.

72. The method of claim 61, in which the individual is suffering from Type 2 diabetes.

73. A method of treatment of a human or animal patient suffering from a condition selected from the group consisting of: hepatic insulin resistance, adipose tissue insulin resistance, muscle insulin resistance, neuronal loss or dysfunction due to glucocorticoid potentiated neurotoxicity, obesity and any combination of the aforementioned conditions, the method comprising the step of administering to said patient a medicament comprising a pharmaceutically active amount of a first agent which is an antagonist of 11β-HSD1, simultaneously or sequentially with a second agent which comprises a diuretic, including an antikaliuretic-diuretic.

74. A method of using a composition according to claim 1, for any one or more of the purposes as set out in Table 2.
